(19) United States
(12) Patent Application Publication
(10) Pub. No.: US 2026/0043815 A1
(43) Pub. Date: Feb. 12, 2026

(54) METHODS FOR DETECTING B-ISOX PRECIPITATES OR CAPTURED PROTEINS AS BIOFLUID BIOMARKERS FOR DIFFERENTIAL DIAGNOSTIC, PATHOPHYSIOLOGY MONITORING OR PRESYMPTOMATIC DIAGNOSTIC OF PREDIABETES, DIABETES AND CANCERS

(71) Applicant: YEEFAN MED INC, Temple City, CA (US)

(72) Inventors: I-Fan WANG, Caotun Township (TW); Hsiang-Yu CHANG, Taichung City (TW); Chen-Hung TING, Yilan City (TW)

(21) Appl. No.: 19/297,512

(22) Filed: Aug. 12, 2025

Related U.S. Application Data
(60) Provisional application No. 63/682,115, filed on Aug. 12, 2024.
(60) Provisional application No. 63/716,876, filed on Nov. 6, 2024.

(51) Int. Cl. G01N 33/68; G01N 33/574

(52) U.S. Cl. CPC G01N 33/6893; G01N 33/57488; G01N 2570/00; G01N 2800/042; G01N 2800/28

(57) ABSTRACT

Described herein are detecting methods for conformational disease, aging and proteinopathies, by measuring the presence of b-isox-precipitates and the levels of b-isox-captured proteins in biofluids of healthy individuals and patients. Research identified additional biomarkers, which made it possible to detect, diagnose or treat, a human disease in a human subject by, with or without adding an isoxazole to an obtained biofluid sample, detecting the biomarker. Use of b-iso and/or biomarkers for diagnosing the disease are made possible.

### CROSS REFERENCE

The present application claims priority benefit to U.S. provisional application No. 63/682,115, filed 12 Aug. 2024, and U.S. provisional application No. 63/716,876 filed 6 Nov. 2024, each application of which is hereby incorporated herein by reference in its entirety.

### TECHNOLOGY FIELD

A method detects, and makes it possible to diagnose or treat, a human disease in a human subject by adding an isoxazole to an obtained biofluid sample to form a biofluid isoxazole composition in the biofluid sample, and detecting a presence of the biofluid isoxazole composition. Research identified additional biomarkers, which made it possible to detect, diagnose or treat, a human disease in a human subject by, with or without adding an isoxazole to an obtained biofluid sample, detecting the biomarker.

Although usable for other purposes, the methods relate to methods for differential diagnostics, real-time pathophysiology monitoring, presymptomatic diagnostics, and pharmacoresponse measurement of conformational diseases and proteinopathies, such as neurodegenerative diseases, diabetes, cancer, psychiatric disorders, and even aging.

### INTRODUCTION

Conformational diseases include more than 50 disorders caused by the accumulation of unfolded or misfolded proteins. Improper protein folding can lead to deposit amorphous aggregates, such as p-TDP-43 aggregates, or ordered amyloid fibrils, such as synuclein and tau inclusions (1).

Proteinopathies refer to pathologies caused by certain misfolded aggregation-prone proteins, such as synuclein, TDP-43 and tau. For example, synucleinopathies and tauopathy.

In patients with Type 2 diabetes (T2D), pancreatic islet amyloid deposits, primarily composed of misfolded Insulin-degrading enzyme-Associated Peptide (IAPP), are frequently observed (2). These aggregates contribute to the reduction of β cell mass and exacerbate insulin secretion defects (3). Both in vitro and in vivo studies, including isolated islet cultures and transgenic mouse models overexpressing human IAPP, have demonstrated the prion-like properties of IAPP aggregates (4). When pancreatic tissue homogenates containing preformed IAPP aggregates are administered, they induce IAPP aggregation and diabetic pathology in both ex vivo and in vivo models (5). Notably, the pathological changes are absent when these aggregates are neutralized with antibodies specific to IAPP (6, 7).

Cancer research has revealed prion-like properties in certain tumor suppressor proteins, notably p53. In tumor tissues, mutant forms of p53 have been observed forming amyloid aggregates that compromise its functionality, potentially accelerating cancer progression (8). A promising therapeutic strategy involves reactivating mutant p53 to restore its essential downstream functions. Development is underway for small molecules designed to stabilize the native conformation of mutant p53, thereby preventing its misfolding and aggregation (9). However, extensive research is still required to optimize these molecules for specific p53 mutations and to assess their long-term safety and efficacy.

b-isox, biotinylated isoxazole (6-(5-(Thiophen-2-yl) isoxazole-3-carboxamido) hexyl 5-((3aS,4S,6aR)-2-oxohexahydro-1H-thieno[3,4-d]imidazol-4-yl) pentanoate), is a small molecule known to precipitate RNA-binding proteins enriched in stress granules and RNA granules. Most of the proteins precipitated by b-isox contained low-complexity (LC) domain, which is responsible for the interaction with b-isox. The LC domains are intrinsic disordered structure and self-interactions. This novel type of self-interaction domain transiently forms a cross-β polymeric condensed phase to perform crucial biological processes, including DNA transcription and replication, chromatin remodeling, nuclear pore passage, signal transduction, synaptic transmission, and cytoskeleton regulation by homotypic- or heterotypic cross-β multimeric interactions. In contrast to pathological cross-β aggregate stacked in-register, the physiological cross-β multimers are loose and reversible (10, 11, 12, 13).

Currently, the cost-effective biomarker testing and noninvasive diagnostic techniques for detection of pathophysiology in clinical and asymptomatic stage of conformational diseases and proteinopathies, particularly prediabetes and early lung cancer, are unmet medical need. The present invention addresses this need and other needs.

### BRIEF SUMMARY

Methods for advances made in the differential diagnostics, real-time monitoring pathophysiology, and asymptomatic diagnostics of diabetes and cancers by detecting the presence of b-isox or its analogs precipitates or the level of b-isox or its analogs-captured proteins from plasma and CSF of patients. Example of neurodegenerative diseases includes, but not limited to, prediabetes, diabetes, lung cancer, pancreatic cancer, Alzheimer's disease (AD), Parkinson's disease (PD) and dementia with Lewy body (DLB).

Example of b-isox precipitated proteins for prediabetes diagnosis includes, but not limited to, IAPP, p-TDP-43, α-synuclein, amyloid β and KRT1.

Example of b-isox precipitated proteins for diabetes diagnosis includes, but not limited to, IAPP, p-TDP-43, α-synuclein, amyloid β and KRT1.

Example of b-isox precipitated proteins for lung cancer diagnosis includes, but not limited to, Rb, CD36, LGALS3BP, LEI, PPP1CB and SAA1.

Example of b-isox precipitated proteins for pancreatic cancer diagnosis includes, but not limited to, p53, CD36, LGALS3BP, LEI, PPP1CB and SAA1.

A method for detecting a human disease in a subject, comprises detecting a presence of biofluid isoxazole-precipitates in a biofluid sample of the subject. In some embodiments, the method of Claim 1, wherein the isoxazole in the isoxazole-precipitates is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate) or its salt or an analog, specifically OG3 or OG4, thereof. OG3 (6-(5-(5-chlorothiophen-2-yl)isoxazole-3-carboxamido)hexyl 5-((3aS,4S,6aR)-2-oxohexahydro-1H-thieno[3,4-d]imidazol-4-yl)pentanoate) and OG4 (6-(5-(benzo[b]thiophen-2-yl)isoxazole-3-carboxamido)hexyl5-((3aS,4S,6aR)-2-oxohexahydro-1H-thieno[3,4-d]imidazol-4-yl)pentanoate).

In some embodiments, the human disease is prediabetes, diabetes, lung cancer, pancreatic cancer, AD, PD or DLB.

In some embodiments, the biofluid is cerebrospinal fluid (CSF) or plasma.

A method for detecting a human conformational disease in a subject, comprises detecting a presence of biofluid isoxazole-captured proteins in a biofluid sample of the subject.

In some embodiments, the isoxazole is biotin-isoxazole (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate) or its analogous, specifically OG3 or OG4. OG3 (6-(5-(5-chlorothiophen-2-yl)isoxazole-3-carboxamido)hexyl 5-((3aS,4S,6aR)-2-oxohexahydro-1H-thieno[3,4-d]imidazol-4-yl)pentanoate) and OG4 (6-(5-(benzo[b]thiophen-2-yl)isoxazole-3-carboxamido)hexyl5-((3aS,4S,6aR)-2-oxohexahydro-1H-thieno[3,4-d]imidazol-4-yl)pentanoate).

In some embodiments, the human conformational disease is prediabetes, diabetes, lung cancer, pancreatic cancer, AD, PD or DLB.

In some embodiments, the isoxazole-captured proteins are detected by a polypeptide.

In some embodiments, the polypeptide is (a) an antibody, or (b) an immunoglobulin chain, or a binding domain thereof which binds to the isoxazole-captured proteins. In some embodiments, the human diseases is prediabetes, and wherein polypeptide for detecting prediabetes is an antibody against IAPP, phospho-TDP-43, α-synuclein, amyloid β, IGKV1-5, SERPINA10, IGHV3-74, APOC3, PARVB, IGHV3-23, CFL1, IGLC2, TTR, SAA4, HBA1, HBB, or CLEC3B. In some embodiments, the human diseases is diabetes, and wherein polypeptide for detecting diabetes is an antibody against IAPP, phospho-TDP-43, α-synuclein, amyloid β, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE. In some embodiments, the human diseases is lung cancer, and wherein polypeptide for detecting lung cancer is an antibody against Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1. In some embodiments, the human diseases is pancreatic cancer, and wherein polypeptide for detecting pancreatic cancer is an antibody against p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1. In some embodiments, the human diseases is AD, and wherein polypeptide for detecting AD is an antibody against FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7; In some embodiments, the human diseases is PD, and wherein polypeptide for detecting PD is an antibody against FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7. In some embodiments, the human diseases is DLB, and wherein the polypeptide for detecting DLB is an antibody against IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE. In some embodiments, the biofluid is plasma and CSF.

A method for detecting conformational disease in a subject, comprises the step of detecting the biofluid level of proteins with cross-β structure in the sample of the subject.

In some embodiments, the biofluid is plasma and CSF. In some embodiments, the proteins with cross-β structure is detected by a combination of a cross-β recognized probe and a polypeptide. In some embodiments, the conformational disease is prediabetes, diabetes, lung cancer, pancreatic cancer, AD, PD or DLB.

The use of a reagent for detecting a b-isox captured complex in the manufacture of a kit to evaluate the risks of developing conformational disease or the progression of conformational diseases.

A method for detecting aging and conformational disease in a subject, comprises the step of detecting the presence of low complexity protein′ complex in the sample of the subject.

In one aspect, a method for detecting a human disease in a human subject, comprising,
optionally obtaining a biofluid sample from the subject,
adding an isoxazole to the obtained biofluid sample to form a biofluid isoxazole composition in the biofluid sample, and
detecting a presence of the biofluid isoxazole composition.

In one embodiment, the isoxazole is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate), or its salt or an analog thereof, especially biotin-isoxazole and very especially biotin-isoxazole, such as OG3 6-(5-(5-chlorothiophen-2-yl)isoxazole-3-carboxamido)hexyl 5-((3aS,4S,6aR)-2-oxohexahydro-1H-thieno[3,4-d]imidazol-4-yl)pentanoate) and OG4 (6-(5-(benzo[b]thiophen-2-yl)isoxazole-3-carboxamido)hexyl5-((3aS,4S,6aR)-2-oxohexahydro-1H-thieno[3,4-d]imidazol-4-yl)pentanoate).

In one embodiment, the biofluid sample is urine, whole blood, plasma, or serum, cerebrospinal fluid (CSF), saliva, or mucosa, such as, urine, saliva, CSF or plasma, and especially CSF or plasma.

In one embodiment, the human disease is prediabetes, diabetes, lung cancer, and pancreatic cancer, Alzheimer's disease (AD), parkinson disease dementia (PD), and dementia with Lewy body (DLB).

In one embodiment, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

In one embodiment, the concentration of isoxazole ranges from 0.075 mM to 0.225 mM, preferably from 0.100 mM to 0.200 mM, in the biofluid sample.

In one embodiment, the biofluid isoxazole composition is in a precipitate.

In one embodiment, the human disease is prediabetes, diabetes, lung cancer, and pancreatic cancer, Alzheimer's disease (AD), parkinson disease dementia (PD), and dementia with Lewy body (DLB).

In one embodiment, further comprising adding a polypeptide to biofluid isoxazole composition in the biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

In one embodiment, the polypeptide is (a) an antibody, or (b) an immunoglobulin chain, or a binding domain thereof which binds to the biofluid isoxazole composition.

In one embodiment, the human disease is prediabetes, and wherein the polypeptide for detecting prediabetes is an antibody against IAPP, p-TDP-43, amyloid β, α-synuclein, IGKV1-5, SERPINA10, IGHV3-74, APOC3, PARVB, IGHV3-23, CFL1, IGLC2, TTR, SAA4, HBA1, HBB, or CLEC3B.

In one embodiment, the human diseases is diabetes, and wherein the polypeptide for detecting diabetes is an antibody against IAPP, p-TDP-43, amyloid β, α-synuclein, IAPP, phospho-TDP-43, amyloid beta, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE.

In one embodiment, the human diseases is lung cancer, and wherein the polypeptide for detecting lung cancer is an antibody against Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1.

In one embodiment, the human diseases is pancreatic cancer, and wherein the polypeptide for detecting pancreatic cancer is an antibody against p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1.

In one embodiment, the human diseases is AD, and wherein polypeptide for detecting AD is an antibody against FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7.

In one embodiment, the human disease is PD, and wherein polypeptide for detecting PD is an antibody against FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7.

In one embodiment, the human disease is DLB, and wherein the polypeptide for detecting DLB is an antibody against KRT1, IAPP, IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE.

In one embodiment, the biofluid sample is urine, whole blood, plasma, or serum, cerebrospinal fluid (CSF), saliva, or mucosa, such as, urine, saliva or plasma, and especially plasma.

In one embodiment, the method is ELISA, such as direct, indirect, sandwich, or competitive ELISA.

Use of an isoxazole to detect a human disease in any method disclosed herein.

In one aspect, a method for detecting a human disease in a human subject, comprising,
detecting a presence of a biomarker from an obtained biofluid sample, and
optionally obtaining a biofluid sample from the subject to obtain the obtained biofluid sample, and
wherein the human disease is selected from prediabetes, diabetes, lung cancer, pancreatic cancer, Alzheimer's disease (AD), Parkinson disease dementia (PD), and dementia with Lewy body (DLB).
wherein, for prediabetes, the biomarker is selected from IAPP, p-TDP-43, amyloid β, α-synuclein, IGKV1-5, SERPINA10, IGHV3-74, APOC3, PARVB, IGHV3-23, CFL1, IGLC2, TTR, SAA4, HBA1, HBB, or CLEC3B;
wherein, for diabetes, the biomarker is selected from IAPP, p-TDP-43, amyloid (3, α-synuclein, IAPP, phospho-TDP-43, amyloid beta, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE;
wherein, for lung cancer, the biomarker is selected from Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1;
wherein, for pancreatic cancer, the biomarker is selected from p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1;
wherein, for AD, the biomarker is selected from FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7;
wherein, for PD, the biomarker is selected from FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7, and
wherein for DLB, the biomarker is selected from KRT1, IAPP, IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE.

In one embodiment, the obtained biofluid sample is urine, whole blood, plasma, or serum, cerebrospinal fluid (CSF), saliva, or mucosa, such as, urine, saliva, CSF or plasma, and especially CSF or plasma.

In one embodiment, the human disease is prediabetes.

In one embodiment, the human disease is diabetes.

In one embodiment, the human disease is lung cancer.

In one embodiment, the human disease is pancreatic cancer.

In one embodiment, the human disease is AD.

In one embodiment, the human disease is PD.

In one embodiment, the human disease is DLB.

In one embodiment, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

In one embodiment, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

In one embodiment, the polypeptide is (a) an antibody, or (b) an immunoglobulin chain, or a binding domain thereof which binds to the biomarker.

In one embodiment, the human disease is prediabetes, and wherein the polypeptide for detecting prediabetes is an antibody against IAPP, p-TDP-43, amyloid β, α-synuclein, IGKV1-5, SERPINA10, IGHV3-74, APOC3, PARVB, IGHV3-23, CFL1, IGLC2, TTR, SAA4, HBA1, HBB, or CLEC3B.

In one embodiment, the human diseases is diabetes, and wherein the polypeptide for detecting diabetes is an antibody against IAPP, p-TDP-43, amyloid β, α-synuclein, IAPP, phospho-TDP-43, amyloid beta, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE.

In one embodiment, the human disease is lung cancer, and wherein the polypeptide for detecting lung cancer is an antibody against Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1.

In one embodiment, the human disease is pancreatic cancer, and wherein the polypeptide for detecting pancreatic cancer is an antibody against p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1.

In one embodiment, the human disease is AD, and wherein polypeptide for detecting AD is an antibody against FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7.

In one embodiment, the human disease is PD, and wherein polypeptide for detecting PD is an antibody against FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7.

In one embodiment, the human disease is DLB, and wherein the polypeptide for detecting DLB is an antibody against KRT1, IAPP, IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE.

In one embodiment, the biofluid is plasma or CSF.

In one embodiment, the method is ELISA, such as direct, indirect, sandwich, or competitive ELISA.

In one embodiment, use of the biomarker to detect the human disease in any method disclosed herein.

In one aspect, a method for detecting conformational disease in a human subject, comprising,
detecting a presence of amyloid oligomers from an obtained biofluid sample, and
optionally obtaining a biofluid sample from the subject to obtain the obtained biofluid sample.

In one embodiment, the biofluid sample is urine, whole blood, plasma, or serum, cerebrospinal fluid (CSF), saliva, or mucosa, such as, urine, saliva or plasma, and especially plasma.

In one embodiment, the amyloid oligomer is selected from A11 recognized oligomers.

In one embodiment, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

In one embodiment, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

In one embodiment, the polypeptide is (a) an antibody, or (b) an immunoglobulin chain, or a binding domain thereof which binds to the biomarker.

In one embodiment, the human disease is prediabetes, and wherein the polypeptide for detecting prediabetes is an antibody against A11.

In one embodiment, the human disease is diabetes, and wherein the polypeptide for detecting diabetes is an antibody against A11.

In one embodiment, the human diseases is lung cancer, and wherein the polypeptide for detecting lung cancer is an antibody against A11.

In one embodiment, the human disease is pancreatic cancer, and wherein the polypeptide for detecting pancreatic cancer is an antibody against A11.

In one embodiment, the human disease is AD, and wherein the polypeptide for detecting AD is an antibody against A11.

In one embodiment, the human disease is PD, and wherein the polypeptide for detecting PD is an antibody against A11.

In one embodiment, the human disease is DLB, and wherein the polypeptide for detecting DLB is an antibody against A11.

In one embodiment, the human disease is ALS, and wherein the polypeptide for detecting ALS is an antibody against A11.

In one embodiment, the biofluid is plasma or CSF.

In one embodiment, the method is ELISA, such as direct, indirect, sandwich, or competitive ELISA.

In one embodiment, use of A11 recognized oligomers to detect conformational diseases in any method disclosed herein.

In one aspect, a method for detecting conformational disease in a human subject at the asymptomatic and prodromal stage, comprising,
detecting a presence of cross β-, amyloid oligomers from an obtained biofluid sample, and
optionally obtaining a biofluid sample from the subject to obtain the obtained biofluid sample.

In one embodiment, the biofluid sample is urine, whole blood, plasma, or serum, cerebrospinal fluid (CSF), saliva, or mucosa, such as, urine, saliva, CSF or plasma, and especially CSF or plasma.

In one embodiment, the cross β-, amyloid oligomer is A11 recognized proteins.

In one embodiment, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

In one embodiment, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

In one embodiment, the polypeptide is (a) an antibody, or (b) an immunoglobulin chain, or a binding domain thereof which binds to the biomarker.

In one embodiment, the polypeptide for detecting sporadic or SOD1 inherited ALS is an antibody against amyloid oligomers.

In one embodiment, the biofluid is plasma or CSF.

In one embodiment, the method is ELISA, such as direct, indirect, sandwich, or competitive ELISA.

In one embodiment, use of A11 recognized protein to detect sporadic or SOD1 inherited ALS in any method disclosed herein.

In one embodiment, use of any described substance or composition for diagnosing a human disease disclosed herein.

In one aspect, a method for detecting conformational disease in a human subject, comprising,
detecting a presence of LBP from an obtained biofluid sample, and
optionally obtaining a biofluid sample from the subject to obtain the obtained biofluid sample.

In one embodiment, the biofluid sample is urine, whole blood, plasma, or serum, cerebrospinal fluid (CSF), saliva, or mucosa, such as, urine, saliva or plasma, and especially plasma.

In one embodiment, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

In one embodiment, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

In one embodiment, the polypeptide is (a) an antibody, or (b) an immunoglobulin chain, or a binding domain thereof which binds to the biomarker.

In one embodiment, the human disease is prediabetes, and wherein the polypeptide for detecting prediabetes is an antibody against LBP.

In one embodiment, the human disease is diabetes, and wherein the polypeptide for detecting diabetes is an antibody against LBP.

In one embodiment, the human disease is lung cancer, and wherein the polypeptide for detecting lung cancer is an antibody against LBP.

In one embodiment, the human disease is pancreatic cancer, and wherein the polypeptide for detecting pancreatic cancer is an antibody against LBP.

In one embodiment, the human disease is AD, and wherein the polypeptide for detecting AD is an antibody against LBP.

In one embodiment, the human disease is PD, and wherein the polypeptide for detecting PD is an antibody against LBP.

In one embodiment, the human disease is DLB, and wherein the polypeptide for detecting DLB is an antibody against LBP.

In one embodiment, the human disease is ALS, and wherein the polypeptide for detecting ALS is an antibody against LBP.

In one embodiment, the biofluid is plasma or urine.

In one embodiment, the method is ELISA, such as direct, indirect, sandwich, or competitive ELISA.

In one embodiment, use of LBP to detect conformational diseases in any method disclosed herein.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

Illustrative embodiments of the present invention are described in detail below with reference to the following Figures:

FIG. 1 comprises a series of images that illustrate the analysis of chemical precipitates, specifically b-isox and its analogs, extracted from the plasma of healthy controls, prediabetic patients, diabetic and cancer patients. Panel (a) analyzes the size of chemical precipitates isolated from the plasma of healthy, prediabetic, and diabetic individuals using b-isox. Panel (b) analyzes the size of chemical precipitates isolated from the plasma of healthy, prediabetic, and diabetic individuals using OG3. Panel (c) analyzes the size of chemical precipitates isolated from the plasma of healthy, prediabetic, and diabetic individuals using OG4. Panel (d) analyzes the size of chemical precipitates isolated from the plasma of healthy and patients with lung cancer using b-isox. Panel (e) analyzes the size of chemical precipitates isolated from the plasma of healthy and patients with NSCL and pancreatic cancer using OG3. Panel (f) analyzes the size of chemical precipitates isolated from the plasma of healthy and patients with lung cancer using OG4. Panel (g) analyzes the size of chemical precipitates isolated from the plasma of healthy and patients with DLB using b-isox, OG3 and OG4. Panel (h) shows the size of b-isox precipitates exhibited a progressive decrease with disease advancement.

FIG. 2 is an assembly of images illustrating a validation of known and newly identified prediabetes and diabetes biomarkers by b-isox-ELISA. Panel (a) contains an assembly of images illustrating plasma levels of IAPP in healthy individuals (H) and patients with prediabetes and diabetes. Panel (b) contains an assembly of images illustrating plasma levels of p-TDP-43, α-synuclein, and amyloid β in healthy individuals (H) and patients with prediabetes and diabetes. Panel (c) contains an assembly of images illustrating plasma levels of KRT1 in healthy individuals (H) and patients with prediabetes and diabetes.

FIG. 3 is an assembly of images illustrating a validation of known and newly identified biomarkers of lung cancer and pancreatic cancer by b-isox-ELISA. Panel (a) contains an assembly of images illustrating plasma levels of Rb in healthy individuals (H) and patients with cancer (NSCLC). Panel (b) contains an assembly of images illustrating plasma levels of p53 in healthy individuals (H) and patients with cancer. Panel (c) contains an assembly of images illustrating plasma levels of CD36, LGALS3BP, LEI, PPP1CB and SAA1 in healthy individuals (H) and patients with cancer.

FIG. 4 is an assembly of images illustrating a validation of known and newly identified biomarkers of DLB by b-isox-ELISA. Panel (a) contains an assembly of images illustrating plasma levels of b-isox captured proteins, including α-synuclein (αSYN), CHL1, RUVBL1, BGN, NCAM, ANXA5, ANK1, DISC1, TDP-43, pTDP-43, Poly(GR), CD14, CA1, STOM, NONO, and PRDX2 in healthy individuals (H) and patients with DLB. Panel (b) contains an assembly of images illustrating plasma levels of IAPP and KRT1 in healthy individuals (H) and patients with DLB.

FIG. 5 is an assembly of images illustrating a validation of A11 recognized oligomers in diabetic and cancer patients by b-isox-ELISA. Panel (a) contains an assembly of images illustrating plasma levels of A11 recognized oligomers in healthy individuals (H), and patients with prediabetes or diabetes. Panel (b) contains an assembly of images illustrating plasma levels of A11 recognized oligomers in healthy individuals (H) and patients with lung or pancreatic cancer. Panel (c) contains an assembly of images illustrating plasma levels of A11 recognized oligomers in healthy individuals (H) and patients with DLB. Panel (d) presents a series of images illustrating the immunodepletion of plasma amyloid oligomers in patients with DLB using A11 antibodies, followed by b-isox ELISA detection of Amyloid oligomers of α-synuclein and p-TDP-43. After the immunodepletion process, α-synuclein and p-TDP-43 levels were reduced to those typical of healthy individuals. Panel (e) presents an analysis illustrating elevated A11 levels were noted in both a patient with SOD1-mutated ALS (son) and a carrier of the inherited SOD1 mutation (asymptomatic mother), but not in a non-mutated individual (patient's brother) and health.

FIG. 6 is an assembly of images illustrating a validation of LBP level in prediabetes, diabetes, cancer and ALS patients by b-isox-ELISA (panel a, c, and d) or OG3-ELISA (panel b). Panel (a) contains an assembly of images illustrating plasma levels of LBP in healthy individuals (H) and patients with prediabetes and diabetes. Panel (b) contains an assembly of images illustrating plasma levels of LBP in healthy individuals (H) and patients with cancer. Panel (c) contains an assembly of images illustrating CSF, plasma and urine levels of SOD1 in a SOD1-mutated ALS patient treating with tofersen. Panel (d) contains an assembly of images illustrating urine levels of LBP in a SOD1-mutated ALS patient treating with tofersen.

### DESCRIPTION OF EMBODIMENTS

Reference will now be made in detail to the present embodiment(s) (exemplary embodiments) of the invention, an example(s) of which is (are) illustrated in the accompanying drawings. Wherever possible, the same reference numbers will be used throughout the drawings to refer to the same or like parts.

The present application is related to International Application no. PCT/US2023/025073, filed 12 Jun. 2024, and U.S. Provisional Application No. 63/351,813, filed 13 Jun. 2022, the entire contents of each of which application is hereby incorporated herein by reference in its entirety.

Table 1. Differential expression of plasma proteins precipitated from healthy and pre-diabetic subjects using b-isox and analyzed by proteomics.

Table 2. Differential expression of plasma proteins precipitated from healthy, and diabetic patients using b-isox and analyzed by proteomics.

Table 3. Differential expression of plasma proteins precipitated from healthy and patients with lung cancer using b-isox and analyzed by proteomics.

Table 4. Differential expression of plasma proteins precipitated from healthy and patients with pancreatic cancer using b-isox and analyzed by proteomics.

Table 5. Differential expression of plasma proteins precipitated from healthy and AD patient using b-isox and analyzed by proteomics.

Table 6. Differential expression of plasma proteins precipitated from healthy and PD patient using b-isox and analyzed by proteomics.

Table 7. Differential expression of plasma proteins precipitated from healthy and DLB patient using b-isox and analyzed by proteomics.

Methods for the detection of conformational diseases and proteinopathies, such as prediabetes, diabetes, lung cancer, and pancreatic cancer, are described herein by using a small-molecule to generate visual precipitates.

In addition, applicants identified novel and specific biofluid biomarkers for differential diagnosis and monitoring pathophysiology in patients with prediabetes, diabetes, lung cancer, pancreatic cancer, AD, PD and DLB.

Also described are novel methods for the plasma diagnosis of conformational diseases. The novel method named b-isox-ELISA, is combination of b-isox chemical-precipitation and immunoassay with specific biomarkers of prediabetes, diabetes, lung cancer, pancreatic cancer, AD, PD and DLB. b-isox ELISA can be used to screen the risks of prediabetes, diabetes, lung cancer, pancreatic cancer, AD, PD and DLB. This invention can be used for real-time readout of pharmacoresponse, and monitoring the relief of pathological burden of misfolded disease proteins in clinical trial, preclinical diagnosis and clinical practice.

### Definitions

As employed above and throughout the disclosure, the following terms, unless otherwise indicated, shall be understood to have the following meanings.

As used herein, the singular forms “a,” “an,” and “the” include the plural reference unless the context clearly indicates otherwise.

### REPRESENTATIVE EMBODIMENTS

Embodiment 1. A method for detecting a human disease in a human subject, comprises, optionally obtaining a biofluid sample from the subject, adding an isoxazole to the obtained biofluid sample to form a biofluid isoxazole composition in the biofluid sample, and detecting a presence of the biofluid isoxazole composition.

Embodiment 2. The method of Embodiment 1, wherein the isoxazole is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate), or its salt or an analog thereof, especially biotin-isoxazole or its salt and very especially biotin-isoxazole.

Embodiment 3. The method of Embodiments 1-2, wherein the biofluid sample is urine, whole blood, plasma, or serum, cerebrospinal fluid (CSF), saliva, or mucosa, such as, urine, saliva, CSF or plasma, and especially CSF or plasma. In some embodiments, the biofluid sample is in the form of a biological fluid such as urine, whole blood, plasma, or serum, cerebrospinal fluid (CSF), saliva, or mucosa, and optionally, the biofluid sample is further processed, e.g., to remove some components, e.g., by techniques to enrich components such as proteins by chemical precipitation. In some embodiments, the biofluid sample is blood, plasma, or serum, CSF, urine or saliva. In some embodiments, the biofluid sample is plasma or CSF.

Embodiment 4. The method of Embodiments 1-3, wherein the human disease is dementia with Lewy body (DLB), prediabetes, diabetes, cancer, or protienopathies.

Embodiment 5. The method of Embodiments 1-4, further comprises thereafter treating the human disease or thereafter changing an existing treatment based on the detecting the presence of the biofluid isoxazole composition, such as a treatment including pharmaceutical therapy for the human disease. In some embodiments, the method further comprises diagnosing the human disease.

Embodiment 6. The method of Embodiments 1-5, wherein the concentration of isoxazole ranges from 0.075 mM to 0.225 mM, preferably from 0.100 mM to 0.200 mM, in the biofluid sample.

Embodiment 7. The method of Embodiments 1-6, wherein the biofluid isoxazole composition is in a precipitate.

Embodiments 8. The method of Embodiment 7, wherein the human disease is DLB, prediabetes, diabetes, lung cancer or pancreatic cancer.

Embodiment 9. The method of Embodiments 1-6, further comprises adding a polypeptide to biofluid isoxazole composition in the biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 10. The method of Embodiment 12, wherein the human conformational disease is prediabetes, diabetes, lung cancer, pancreatic cancer, AD, PD, or DLB.

Embodiment 11. The method of Embodiments 9-10, wherein the polypeptide is (a) an antibody, or (b) an immunoglobulin chain, or a binding domain thereof which binds to the biofluid isoxazole composition.

Embodiment 12. The method of Embodiment 11, wherein the human diseases is prediabetes, and wherein polypeptide for detecting prediabetes is an antibody against IAPP, phospho-TDP-43, α-synuclein, amyloid β, IGKV1-5, SERPINA10, IGHV3-74, APOC3, PARVB, IGHV3-23, CFL1, IGLC2, TTR, SAA4, HBA1, HBB, or CLEC3B.

Embodiment 13. The method of Embodiment 11, wherein the human diseases is diabetes, and wherein polypeptide for detecting diabetes is an antibody against IAPP, phospho-TDP-43, α-synuclein, amyloid β, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE.

Embodiment 14. The method of Embodiment 11, wherein the human diseases is lung cancer, and wherein polypeptide for detecting lung cancer is an antibody against Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1.

Embodiment 15. The method of Embodiment 11, wherein the human diseases is pancreatic cancer, and wherein polypeptide for detecting pancreatic cancer is an antibody against p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1.

Embodiment 16. The method of Embodiment 11, wherein the human diseases is AD, and wherein polypeptide for detecting AD is an antibody against FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7.

Embodiment 17. The method of Embodiment 11, wherein the human diseases is PD, and wherein polypeptide for detecting PD is an antibody against FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7.

Embodiments 18. The method of Embodiment 11, wherein the human diseases is DLB, and wherein the polypeptide for detecting DLB is an antibody against IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE.

Embodiment 19. The method of Embodiments 9-18, wherein biofluid is plasma or CSF.

Embodiment 20. The method of Embodiments 9-19, wherein the method is ELISA, such as direct, indirect, sandwich, or competitive ELISA.

Embodiment 21. Use of an isoxazole to detect a human disease in any method of Embodiments 1-20.

Embodiment 22. A method for detecting a human disease in a human subject, comprises, detecting a presence of a biomarker from an obtained biofluid sample, and optionally obtaining a biofluid sample from the subject to obtain the obtained biofluid sample, and wherein the human disease is selected from prediabetes, diabetes, lung cancer, and pancreatic cancer, AD, PD or DLB; wherein, for prediabetes, the biomarker is selected from IAPP, phospho-TDP-43, α-synuclein, amyloid β, IGKV1-5, SERPINA10, IGHV3-74, APOC3, PARVB, IGHV3-23, CFL1, IGLC2, TTR, SAA4, HBA1, HBB, or CLEC3B; wherein, for diabetes, the biomarker is selected from α-synuclein, IAPP, phospho-TDP-43, amyloid β, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE; wherein for lung cancer, the biomarker is selected from Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1; wherein for pancreatic cancer, the biomarker is selected from p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1; wherein for AD, the biomarker is selected from FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7; wherein for PD, the biomarker is selected from FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7; and wherein for DLB, the biomarker is selected from IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE.

Embodiment 23. The method of Embodiment 20, wherein the obtained biofluid sample is urine, whole blood, plasma, or serum, cerebrospinal fluid (CSF), saliva, or mucosa, such as, urine, saliva, CSF or plasma, and especially CSF or plasma. The biofluid sample includes those in Embodiment 3.

Embodiment 24. The method of Embodiments s 22-23, wherein the human disease is prediabetes and diabetes.

Embodiment 25. The method of Embodiments 22-23, wherein the human disease is lung cancer and pancreatic cancer.

Embodiment 26. The method of Embodiments 22-23, wherein the human disease is DLB, AD and PD.

Embodiment 27. The method of Embodiments 22-26, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

Embodiment 28. The method of Embodiments 22-27, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 29. The method of Embodiment 28, wherein the polypeptide is (a) an antibody, or (b) an immunoglobulin chain, or a binding domain thereof which binds to the biomarker.

Embodiment 30. The method of Embodiment 29, wherein the human diseases is diabetes, and wherein the polypeptide for detecting prediabetes is an antibody against IAPP, phospho-TDP-43, α-synuclein, amyloid β, IGKV1-5, SERPINA10, IGHV3-74, APOC3, PARVB, IGHV3-23, CFL1, IGLC2, TTR, SAA4, HBA1, HBB, or CLEC3B; and diabetes is an antibody α-synuclein, IAPP, phospho-TDP-43, amyloid β, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE.

Embodiment 31. The method of Embodiment 29, wherein the human diseases is cancer, and wherein polypeptide for detecting lung cancer is an antibody against Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1; and pancreatic cancer is an antibody against p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1.

Embodiment 32. The method of Embodiment 29, wherein the human diseases is neurodegenerative disease, and wherein polypeptide for detecting DLB is an antibody against IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE; AD is an antibody against FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, LlCAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7; and PD is an antibody against FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, LlCAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7.

Embodiment 33. The method of Embodiments 30-32, wherein biofluid is plasma or CSF.

Embodiment 34. The method of Embodiment 28-33, wherein the method is ELISA, such as direct, indirect, sandwich, or competitive ELISA.

Embodiment 35. Use of the biomarker to detect the human disease in any method of Embodiments 22-34.

Embodiment 36. A method for detecting proteinopathies in a human subject, comprises, detecting total amyloid oligomers from an obtained biofluid sample, and optionally obtaining a biofluid sample from the subject to obtain the obtained biofluid sample.

Embodiment 37. The method of Embodiment 34, wherein the biofluid sample is urine, whole blood, plasma, or serum, cerebrospinal fluid (CSF), saliva, or mucosa, such as, urine, saliva, CSF or plasma, and especially CSF or plasma.

Embodiment 38. The method of Embodiments 36-37, wherein the total amyloid oligomers are those that bind to the A11 antibody.

Embodiment 39. The method of Embodiments 36-38, further comprises thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

Embodiment 40. The method of Embodiments 36-39, further comprising adding a polypeptide to the obtained biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

Embodiment 41. The method of Embodiment 38, wherein the polypeptide is (a) an antibody, or (b) an immunoglobulin chain, or a binding domain thereof which binds to the biomarker.

Embodiment 42. The method of Embodiment 40, wherein the human diseases is proteinopathies, and wherein A11 antibody for detecting proteinopathy is an antibody against amyloid oligomers.

Embodiment 43. The method of Embodiment 39-42, wherein biofluid is plasma or CSF.

Embodiment 44. The method of Embodiments 39-42, wherein the method is ELISA, such as direct, indirect, sandwich, or competitive ELISA.

Embodiment 45. Use of total amyloid oligomers to detect proteinopathies in any method of Embodiments 36-44.

Methods for Diagnosing Prediabetes, Diabetes, Lung Cancer, Pancreatic Cancer, and Dementia with Lewy Bodies (DLB) by Detecting b-Isox or its Analogs Precipitates from Plasma Samples

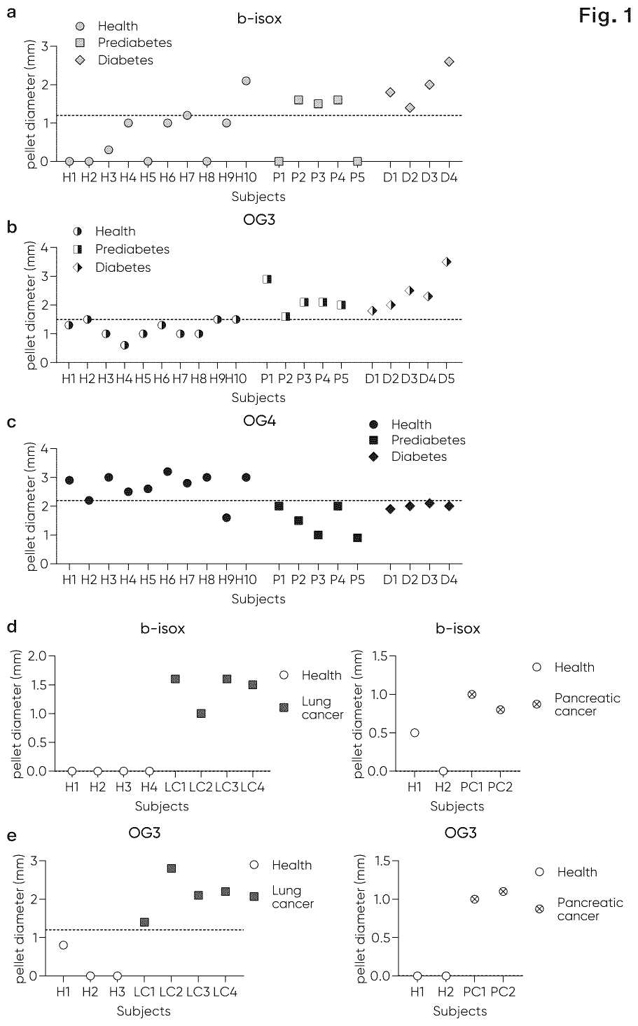

Applicants uses a small-molecule compound, b-isox to generate precipitates, which can be visually observed in samples from patients with prediabetes and diabetes, but not in samples from healthy people (FIG. 1a). b-isox precipitates accurately discriminated healthy individuals from prediabetes and diabetes patients.

Applicants uses a small-molecule compound, OG3 to generate precipitates, which can be visually observed in samples from patients with prediabetes and diabetes, but not in samples from healthy people (FIG. 1b). OG3 precipitates accurately discriminated healthy individuals from prediabetes and diabetes patients.

Applicants uses a small-molecule compound, OG4 to generate precipitates, which can be visually observed in samples from patients with prediabetes and diabetes, but not in samples from healthy people (FIG. 1c). OG4 precipitates accurately discriminated healthy individuals from prediabetes and diabetes patients.

Applicants uses a small-molecule compound, b-isox to generate precipitates, which can be visually observed in samples from patients with lung cancer, but not in samples from healthy people (FIG. 1d). b-isox precipitates accurately discriminated healthy individuals from lung cancer patients.

Applicants uses a small-molecule compound, OG3 to generate precipitates, which can be visually observed in samples from patients with lung cancer, but not in samples from healthy people (FIG. 1e). OG3 precipitates accurately discriminated healthy individuals from lung cancer patients.

Applicants uses a small-molecule compound, OG4 to generate precipitates, which can be visually observed in samples from patients with lung cancer and pancreatic cancer, but not in samples from healthy people (FIG. 1f). OG4 precipitates accurately discriminated healthy individuals from lung cancer and pancreatic cancer patients.

Applicants uses a small-molecule compound, b-isox to generate precipitates, which can be visually observed in samples from patients with DLB, but not in samples from healthy people (FIG. 1g). b-isox precipitates accurately discriminated healthy individuals from DLB patients. These levels progressively decreased as the disease advanced, as illustrated in FIG. 1h.

Methods for Prediabetes and Diabetes Diagnostic by Detecting Plasma b-Isox-Captured Proteins

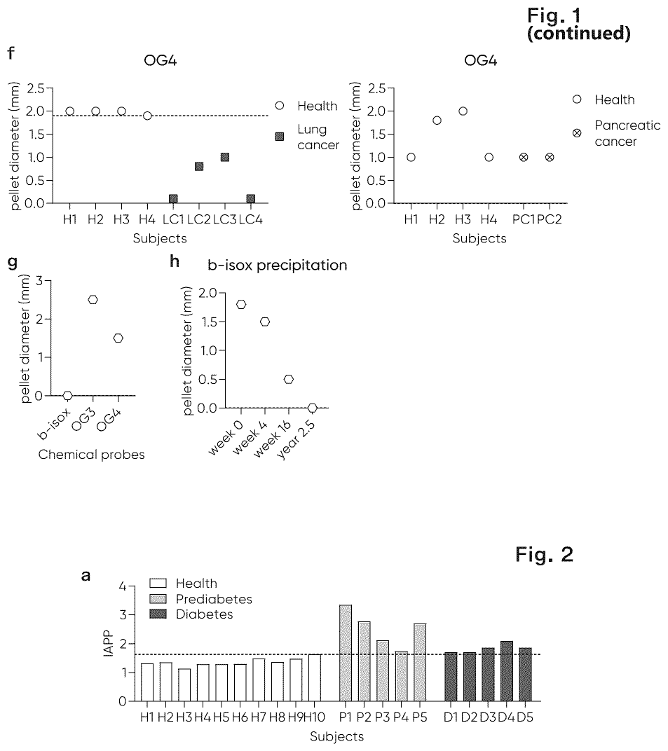

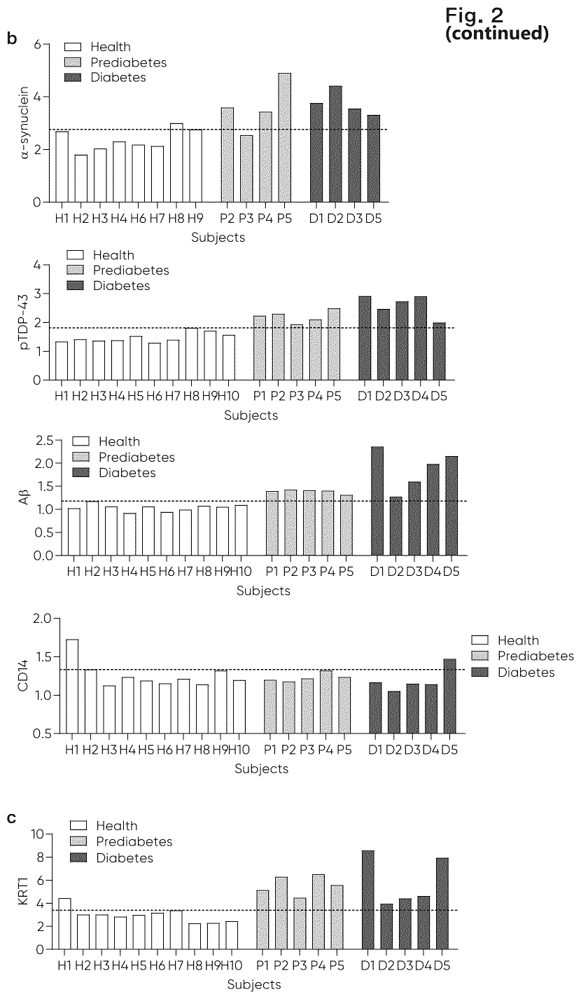

Applicants found b-isox ELISA detected the levels of IAPP tested in plasma, and significantly differed between prediabetes and diabetes groups and HCs (FIG. 2a).

Applicants further identified novel biofluid biomarkers of PD by proteomics analysis of b-isox precipitates and further replicated and validated the identified biomarker candidates, including p-TDP-43, amyloid β, α-synuclein and KRT1 in another cohort of patients (FIGS. 2b and c, and Table 1, 2). The levels of these novel biomarkers tested in the plasma significantly differed between the disease groups and healthy individuals.

Methods for Lung and Pancreatic Cancers Diagnostic by Detecting Plasma b-Isox-Captured Proteins

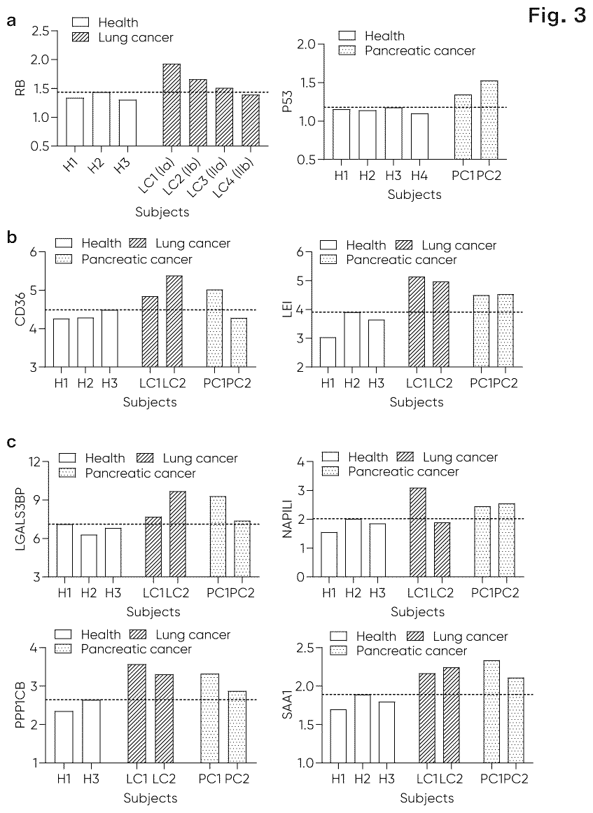

Applicants found tb-isox ELISA detected the levels of Rb and p53 tested in plasma, and significantly differed between cancer groups, including lung cancer and pancreatic cancer, and HCs (FIGS. 3a and b).

Applicants further identified novel biofluid biomarkers of cancer by proteomics analysis of b-isox precipitates and further replicated and validated the identified biomarker candidates, including CD36, LGALS3BP, LEI, PPP1CB and SAA1 in another cohort of patients (FIG. 3c and Table 3, 4). The levels of these novel biomarkers tested in the plasma significantly differed between the disease groups and healthy individuals.

Methods for AD, PD or DLB Diagnostic by Detecting Plasma b-Isox-Captured Proteins

Applicants further identified novel biofluid biomarkers of AD, PD and DLB by proteomics analysis of b-isox precipitates (Table 5, 6, 7).

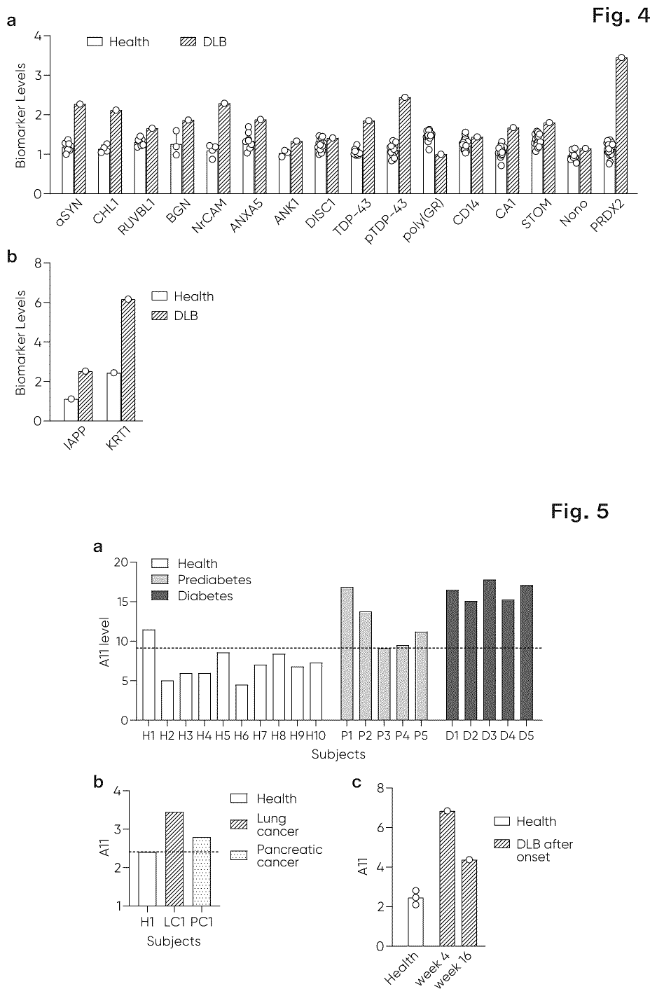

Applicants found b-isox ELISA detected the levels of α-synuclein (αSYN), CHL1, RUVBL1, BGN, NCAM, ANXA5, ANK1, DISC1, TDP-43, pTDP-43, Poly(GR), CD14, CA1, STOM, NONO, PRDX2, and IAPP, KRT1 tested in plasma, and significantly differed between DLB groups and HCs (FIG. 4).

Methods for Diagnostic of Proteinopathies by Detecting Plasma b-Isox-Captured all Recognized Amyloid Oligomers

Applicants found the b-isox ELISA detected the levels of A11 specific-captured amyloid oligomers tested in plasma, and significantly differed between proteinopathies, including diabetes, cancers, and neurodegenerative disease, and health controls (FIG. 5a-e).

Significantly, plasma levels of A11 antibody-recognized proteins were quantitatively assessed in individuals with and without SOD1 mutations using the b-isox-ELISA method. Elevated A11 levels were noted in both a patient with SOD1-mutated ALS (son) and a carrier of the inherited SOD1 mutation (asymptomatic mother), but not in a non-mutated individual (patient's brother) and health, as shown in FIG. 5e. Thus, A11 proteins serve as not only for differential diagnostic of ALS, but also asymptomatic biomarkers for ALS risk assessment.

All of applicants' data suggested plasma b-isox-captured, A11 recognized proteins can act as sensitive indicators of pathophysiological changes during the pathogenesis of diabetes, cancers, and neurodegenerative diseases, and enable a precision medicine approach for proteinopathies.

### Methods for Diagnostic of Proteinopathies by Detecting Plasma or Urine Levels of LBP

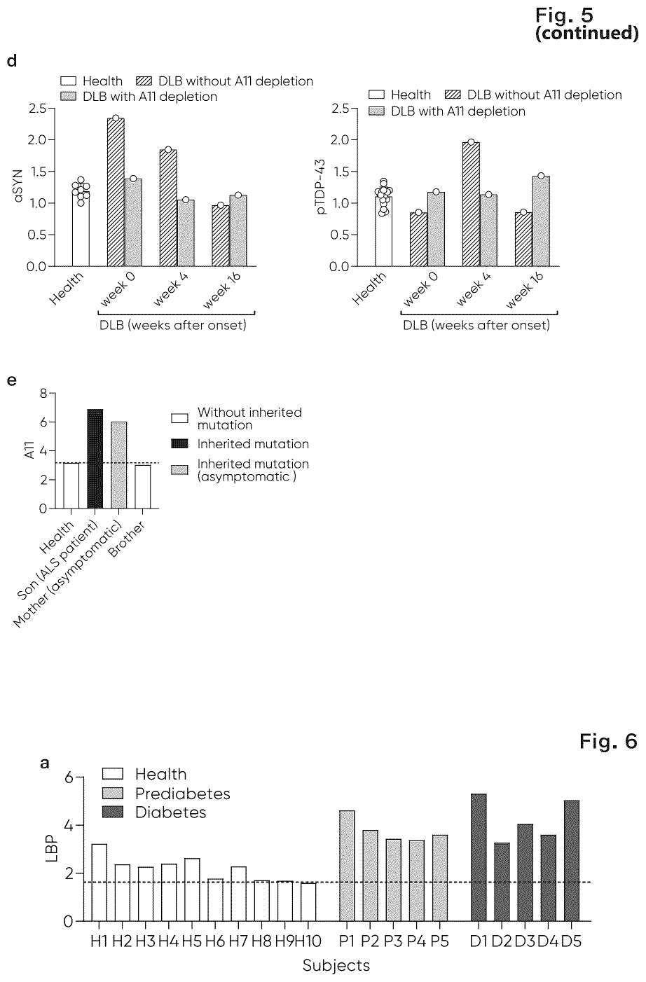

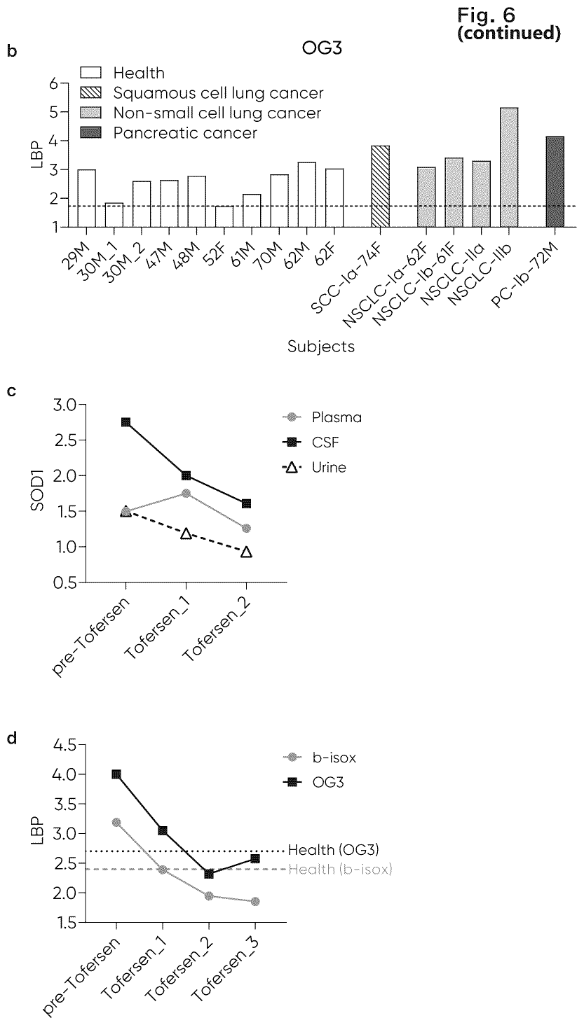

Applicants found the levels of LBP tested in plasma and urine, and significantly differed between proteinopathies, including neurodegenerative disease, cancers, prediabetes, and diabetes, and health controls (FIG. 6a-d).

All of applicants' data suggested plasma or urine LBP can act as sensitive indicators of pathophysiological changes during the pathogenesis of diabetes, cancers, and neurodegenerative diseases, and enable a precision medicine approach for proteinopathies.

### Description of Materials and Methods Used in the Examples

The following materials and methods were used in the Examples described below.

Reagents and Antibodies: b-isox was purchased from Sigma and Dalton dissolved in dimethyl sulfoxide (DMSO). The primary antibodies against SERPINB1 (MBS2025872) were purchased from Mybiosource. The primary antibodies against Rb (#17218-1-AP), p53 (#21891-1-AP), alpha-synuclein (#10842-1-AP phospho-TDP-43 (Ser 409/410) (#22309-1-AP), KRT1 (#16848-1-AP), Galectin-3-binding protein (#10281-1-AP), Nucleosome assembly protein 1-like 1 (#14898-1-AP), SAA1 (#16721-1-AP), CD36 (#18836-1-AP) and CD5L (#17224-1-AP) were purchased from Proteintech. A11 antibody (#AB9234) were purchased from MERCK MILIPORE. IAPP antibody (#PA5-98309) were purchased from Invitrogen.

b-isox Precipitation: 10 mM biotinylated isoxazole was added to the human blood plasma or CSF to a final concentration of 100 to 200 μM. The mixtures were then incubated at 4° C. for 60 min, centrifuged at 15000 rpm for 15 min at 4° C., and the supernatant was discarded. The diameters of b-isox precipitates were measured.

b-isox precipitation and Enzyme-linked immunosorbent assay (ELISA) Blood samples from patients and healthy control were firstly collected through Blood Collection Tubes. Centrifugation of the tubes for 15 min at 2,200′g. The resulting supernatant (upper layer) as the plasma sample. Before immunoassay, gently mixed 50-100 mL plasma (or CSF) with 0.5-1 mL b-isox through pipetting and rotated for 1h at 4° C. Wash each streptavidin-coated microwell 3 times by 200 mL wash buffer (WB/(25 mM Tris, 150 mM NaCl; pH 7.2), 0.1% BSA, 0.05% Tween®-20) (do not allow wells to dry). Add 100 μL of the reaction mixtures to each well and incubate for 2 h with shaking (˜60 rpm) at room temperature (RT). Wash 3 times with 200 μL WB when reaction finished. Add 100 mL primary antibody diluent (appropriate primary antibody dilution in WB), incubating for 1h at RT with shaking. At this step, a no primary antibody control should be included (Add antibody diluent alone in a sample well). Wash 3 times with 200 μL WB. Add 100 μL antibody diluent with appropriately diluted HRP-conjugated secondary antibody, incubating for 1h at RT with shaking. Equilibrate the TMB substrate solution to RT at this step. Wash 3 times with 200 μL WB. Add 100 μL of the TMB Substrate Solution to each microplate well, incubating for 15-30 min until the color develops. Stop the reaction by adding 100 μL 2M sulfuric acid (or 2N HCl). Measure the optical density of at 450 nm by a microplate reader.

### EXAMPLES

### Example 1: Analysis of b-Isox, OG3, and OG4 Precipitates in Plasma from Healthy Controls and Patients with Prediabetes, Diabetes, and Cancers

100 ul of blood plasma from normal individuals and patients with prediabetes and diabetes was incubated with b-isox, and then centrifuged to pull down binding proteins. The statistical analysis of the size of b-isox precipitates is shown in FIGS. 1a and d. The cut-off value is set at 1.2 mm. Values below 1.2 mm are considered within normal limits and suggest an absence of the significant pathological elevations typically associated with prediabetes, diabetes, and cancers. In our analysis, we employed a cut-off value of 1.2 mm for b-isox precipitates measured in plasma samples. This threshold was determined based on prior validation studies that identified it as critical for distinguishing pathological conditions from normal states.

The statistical analysis of the size of OG3 precipitates is shown in FIGS. 1b and e. The cut-off value is set at 1.5 mm. Values below 1.5 mm are considered within normal limits and suggest an absence of the significant pathological elevations typically associated with prediabetes, diabetes, and cancers.

The statistical analysis of the size of OG4 precipitates from the plasma of prediabetes, diabetes, and cancers is shown in FIGS. 1c and f. Values above 2.1 mm are considered as healthy.

The statistical analysis of the size of precipitates from the plasma of patient with DLB using b-isox, OG3 and OG4 is shown in FIG. 1g. These levels gradually decreased with the progression of the disease, as illustrated in FIG. 1h.

### Example 2: Identification and Validation of Novel Plasma Biomarkers of Prediabetes and Diabetes by b-Isox ELISA

The plasma levels of IAPP in patients with prediabetes and diabetes were quantitatively analyzed using the b-isox-ELISA method. Significant differences in IAPP concentrations were observed between these patients and healthy individuals, as illustrated in FIG. 2a.

Furthermore, an examination of other proteins associated with conformational diseases revealed that plasma levels of p-TDP-43, amyloid β, and α-synuclein exhibit a slight increase in patients with prediabetes and a significant increase in those with diabetes, as shown in FIG. 2b.

Proteomic analysis was employed to comprehensively investigate the precipitates isolated from the plasma of patients with prediabetes and diabetes. This analysis yielded several biomarker candidates for prediabetes and diabetes, as detailed in Table 1 and 2.

Subsequent validation of the identified biomarker candidates was conducted in an additional cohort of patients, confirming KRT1 as a biomarker of pathophysiology in prediabetes and diabetes. Significant differences in plasma levels of KRT1 between the disease groups and healthy individuals were observed, as depicted in FIG. 2c.

### Example 3: Identification and Validation of Novel Plasma Biomarkers of Lung and Pancreatic Cancers by b-Isox ELISA

Plasma levels of Rb in patients with lung cancer were quantitatively assessed using the b-isox-ELISA method. A significant increase in the plasma levels of Rb was observed at the Ia stage of lung cancer compared to healthy individuals. These levels gradually decreased with the progression of the disease, as illustrated in FIG. 3a.

Plasma levels of p53 in patients with pancreatic cancer were quantitatively analyzed using the b-isox-ELISA method. Significant differences in the protein concentrations of p53 were observed between these patients and healthy individuals, as depicted in FIG. 3b.

Proteomic analysis was employed to comprehensively investigate the precipitates isolated from the plasma of patients with lung and pancreatic cancers. This analysis yielded several biomarker candidates for lung and pancreatic cancers, as detailed in Table 3 and 4.

Subsequent validation of the identified biomarker candidates was conducted in an additional cohort of patients, confirming CD36, LGALS3BP, LEI, PPP1CB and SAA1 as a biomarker of pathophysiology in lung and pancreatic cancers. Significant differences in plasma levels of CD36, LGALS3BP, LEI, PPP1CB and SAA1 between the disease groups and healthy individuals were observed, as depicted in FIG. 3c.

### Example 4: Identification and Validation of Novel Plasma Biomarkers of DLB by b-Isox ELISA

Plasma levels of b-isox captured proteins in a patient with DLB were quantitatively assessed using the b-isox-ELISA method. A significant increase in the plasma levels of b-isox captured proteins was observed in a patient with DLB compared to healthy individuals (FIG. 4a). These biomarkers include α-synuclein (αSYN), CHL1, RUVBL1, BGN, NCAM, ANXA5, ANK1, DISC1, TDP-43, pTDP-43, Poly(GR), CD14, CA1, STOM, NONO, and PRDX2.

Plasma levels of newly identified proteins, including IAPP and KRT1, in patients with DLB were quantitatively assessed using the b-isox-ELISA method. A significant increase in the plasma levels of IAPP and KRT1 was observed in a patient with DLB compared to healthy individuals (FIG. 4b).

### Example 5: A11 Recognized Oligomers is a Diagnostic and Asymptomatic Biomarker for Diabetes, Cancers, DLB and ALS

Plasma levels of A11 antibody-recognized proteins in patients with prediabetes and diabetes were quantitatively analyzed using the b-isox-ELISA method. Significant differences in the concentrations of A11-positive oligomers were observed between these patients and healthy individuals, as depicted in FIG. 5a.

Plasma levels of A11 antibody-recognized proteins in patients with cancers were quantitatively analyzed using the b-isox-ELISA method. Significant differences in the concentrations of A11-positive oligomers were observed between these patients and healthy individuals, as depicted in FIG. 5b.

Plasma levels of A11 antibody-recognized proteins in patients with DLB were quantitatively analyzed using the b-isox-ELISA method. Significant differences in the concentrations of A11-positive oligomers were observed between these patients and healthy individuals, as depicted in FIG. 5c.

FIG. 5d displays images showing the use of A11 antibodies to immunodeplete plasma amyloid oligomers in DLB patients, followed by b-isox ELISA detection of α-synuclein and p-TDP-43. Post-depletion, α-synuclein and p-TDP-43 levels decreased to those typical in healthy individuals.

Plasma levels of A11 antibody-recognized proteins were quantitatively analyzed in patients with SOD1-mutated ALS, and individuals with and without inherited SOD1 mutations, using the b-isox-ELISA method. Increased levels of A11 were observed in both the SOD1-mutated ALS patient (son) and the individual with an inherited SOD1 mutation (asymptomatic mother). However, the individual without the SOD1 mutation (patient's brother) and health group did not show increased A11 levels, as depicted in FIG. 5e. Thus, A11 antibody-recognized proteins serve as asymptomatic biomarkers of ALS and can be used to assess whether subjects are at risk.

### Example 6: Plasma and Urine LBP is a Diagnostic and Asymptomatic Biomarker for Prediabetes, Diabetes, Cancers, DLB and ALS

Plasma levels of LBP in patients with prediabetes and diabetes were quantitatively analyzed using the b-isox-ELISA method. Significant differences in the concentrations of LBP were observed between these patients and healthy individuals, as depicted in FIG. 6a.

Plasma levels of LBP in patients with cancers were quantitatively analyzed using the b-isox-ELISA method. Significant differences in the concentrations of LBP were observed between these patients and healthy individuals, as depicted in FIG. 6b.

Urine levels of SOD1 and LBP in patients with ALS were quantitatively analyzed using the b-isox-ELISA method with b-isox and OG3. Significant differences in the concentrations of SOD1 and LBP in urine were observed after tofersen treatment, as depicted in FIGS. 6c and d.

### Example 7: Identification of Novel Plasma Biomarkers of AD, PD and DLB

Proteomic analysis was utilized to comprehensively investigate the precipitates isolated from the plasma of patients with AD. This analysis identified several biomarker candidates for AD, as detailed in Table 5.

Proteomic analysis was utilized to comprehensively investigate the precipitates isolated from the cerebrospinal fluid (CSF) of patients with PD. This analysis identified several biomarker candidates for PD, as detailed in Table 6.

Proteomic analysis was utilized to comprehensively investigate the precipitates isolated from the cerebrospinal fluid (CSF) of patients with DLB. This analysis identified several biomarker candidates for DLB, as detailed in Table 7.

Tables are as follows:

TABLE 1
Differential Expression Analysis of Plasma Proteins
in Prediabetes People Compared to Healthy Controls.

| | Gene | Normalized | |
|---|---|---|---|
| Accession | name | ratio | Trend |
| P01602 | IGKV1-5 | 2.1742167119016504 | up-regulated |
| Q9UK55 | SERPINA10 | 8.4566966623946006E−3 | down- |
| A0A0B4J1X5 | IGHV3-74 | 8.4566966623946006E−3 | regulated |
| P02656 | APOC3 | 8.4566966623946006E−3 | |
| Q9HBI1 | PARVB | 8.4566966623946006E−3 | |
| P01764 | IGHV3-23 | 0.21564576489106216 | |
| P23528 | CFL1 | 0.24017018521200645 | |
| P0DOY2 | IGLC2 | 0.35264425082185458 | |
| P02766 | TTR | 0.37124898347912272 | |
| P35542 | SAA4 | 0.40084742179750371 | |
| P69905 | HBA1 | 0.41268679712485612 | |
| P68871 | HBB | 0.41437813645733523 | |
| P05452 | CLEC3B | 0.44735925344067407 | |

TABLE 2
Differential Expression Analysis of Plasma Proteins
in Diabetes Patients Compared to Healthy Controls.

| | Gene | Normalized | |
|---|---|---|---|
| Accession | name | ratio | Trend |
| P08567 | PLEK | 3.1614754178244442 | up- |
| | | | regulated |
| P04264 | KRT1 | 3.0594923398301068 | up- |
| | | | regulated |
| P0DOX2 | Immuno- | 2.0477554549656536 | up- |
| | globulin | | regulated |
| | alpha-2 | | |
| | heavy | | |
| | chain | | |
| P18428 | LBP | 2.4848257892270977 | up- |
| | | | regulated |
| P01768 | IGHV3-30 | 2.8603825208887832 | up- |
| | | | regulated |
| A0A0C4DH34 | IGHV4-28 | 2.0267113277604731 | up- |
| | | | regulated |
| P0C0L5 | C4B | 3.2901683495792033 | up- |
| | | | regulated |
| P04114 | APOB | 2.0930812674075812 | up- |
| | | | regulated |
| P02760 | AMBP | 2.6369910167107116 | up- |
| | | | regulated |
| P07437 | TUBB | 2.0339958333314971 | up- |
| | | | regulated |
| P04406 | GAPDH | 2.0145704851420998 | up- |
| | | | regulated |
| P35908 | KRT2 | 80.938950789156266 | up- |
| | | | regulated |
| P13645 | KRT10 | 80.938950789156266 | up- |
| | | | regulated |
| Q9UK55 | SERPINA10 | 8.093895078915626E−3 | down- |
| | | | regulated |
| A0A0B4J1X5 | IGHV3-74 | 8.093895078915626E−3 | down- |
| | | | regulated |
| P02656 | APOC3 | 8.093895078915626E−3 | down- |
| | | | regulated |
| P01764 | IGHV3-23 | 0.34884687790126362 | down- |
| | | | regulated |
| P0DOY2 | IGLC2 | 0.322137024140842 | down- |
| | | | regulated |
| P05452 | CLEC3B | 8.093895078915626E−3 | down- |
| | | | regulated |
| A0A075B6H7 | IGKV3-7 | 0.40145719591421519 | down- |
| | | | regulated |
| A0A0C4DH25 | IGKV3D-20 | 0.45811446146662455 | down- |
| | | | regulated |
| A0A0C4DH72 | IGKV1-6 | 0.47349286211656422 | down- |
| | | | regulated |
| O43866 | CD5L | 0.45568629294294982 | down- |
| | | | regulated |
| P01871 | IGHM | 0.47430225162445583 | down- |
| | | | regulated |
| P01599 | IGKV1-17 | 0.46863652506921483 | down- |
| | | | regulated |
| A0A0J9YXX1 | IGHV5-10-1 | 5.2610318012951574E−2 | down- |
| | | | regulated |
| P62258 | YWHAE | 8.093895078915626E−3 | down- |
| | | | regulated |

TABLE 3
Differential Expression Analysis of Plasma Proteins
in Lung Cancer Patients Compared to Healthy Controls.

| | Gene | Normalized | |
|---|---|---|---|
| Accession | name | ratio | Trend |
| A0A0J9YXX1 | IGHV5-10-1 | 2.002129075 | up-regulated |
| Q13011 | ECH1 | 2.14582255 | up-regulated |
| P12931 | SRC | 2.167110472 | up-regulated |
| P40197 | GP5 | 2.174029047 | up-regulated |
| Q71U36 | TUBA1A | 2.341139235 | up-regulated |
| Q15942 | ZYX | 2.407663992 | up-regulated |
| P18428 | LBP | 2.440128073 | up-regulated |
| Q14247 | CTTN | 2.453965222 | up-regulated |
| P00488 | F13A1 | 2.486961502 | up-regulated |
| P04792 | HSPB1 | 2.54497109 | up-regulated |
| Q14697 | GANAB | 2.574241982 | up-regulated |
| P16671 | CD36 | 3.002129217 | up-regulated |
| P07900 | HSP90AA1 | 3.357637516 | up-regulated |
| P01137 | TGFB1 | 3.484300653 | up-regulated |
| P31150 | GDI1 | 3.621075553 | up-regulated |
| P50148 | GNAQ | 5.309207777 | up-regulated |
| P0DJI8 | SAA1 | 11.31346621 | up-regulated |
| P02741 | CRP | 17.72219517 | up-regulated |
| P0DJI9 | SAA2 | 53.2198053 | up-regulated |
| O43294 | TGFB1I1 | 53.2198053 | up-regulated |
| P06744 | GPI | 53.2198053 | up-regulated |
| O15143 | ARPC1B | 53.2198053 | up-regulated |
| P55209 | NAP1L1 | 53.2198053 | up-regulated |
| Q9Y251 | HPSE | 53.2198053 | up-regulated |
| Q8N0Y7 | PGAM4 | 53.2198053 | up-regulated |
| P19367 | HK1 | 53.2198053 | up-regulated |
| Q15833 | STXBP2 | 53.2198053 | up-regulated |
| Q27J81 | INF2 | 53.2198053 | up-regulated |
| P40227 | CCT6A | 53.2198053 | up-regulated |
| Q06187 | BTK | 53.2198053 | up-regulated |
| P30740 | SERPINB1 | 53.2198053 | up-regulated |
| A0A0A0MT36 | IGKV6D-21 | 0.005321981 | down-regulated |
| P02655 | APOC2 | 0.005321981 | down-regulated |
| P35858 | IGFALS | 0.005321981 | down-regulated |
| P02656 | APOC3 | 0.149547653 | down-regulated |
| P04114 | APOB | 0.163384802 | down-regulated |
| P02652 | APOA2 | 0.195848884 | down-regulated |
| P19827 | ITIH1 | 0.221926588 | down-regulated |
| P19823 | ITIH2 | 0.222458786 | down-regulated |
| P02766 | TTR | 0.234699341 | down-regulated |
| P08519 | LPA | 0.253326273 | down-regulated |
| P27169 | PON1 | 0.25705166 | down-regulated |
| P04439 | HLA-A | 0.263438036 | down-regulated |
| P05090 | APOD | 0.271953205 | down-regulated |
| P02647 | APOA1 | 0.27621079 | down-regulated |
| P35542 | SAA4 | 0.288451345 | down-regulated |
| P05452 | CLEC3B | 0.292708929 | down-regulated |
| P02765 | AHSG | 0.312400257 | down-regulated |
| P29622 | SERPINA4 | 0.325705208 | down-regulated |
| P25311 | AZGP1 | 0.354443903 | down-regulated |
| P04278 | SHBG | 0.364023468 | down-regulated |
| P05154 | SERPINA5 | 0.365620062 | down-regulated |
| Q06033 | ITIH3 | 0.367748855 | down-regulated |
| P02787 | TF | 0.377860618 | down-regulated |
| P00739 | HPR | 0.381053806 | down-regulated |
| P02649 | APOE | 0.386375786 | down-regulated |
| O95445 | APOM | 0.391165569 | down-regulated |
| A0A0A0MRZ8 | IGKV3D-11 | 0.398616342 | down-regulated |
| P22352 | GPX3 | 0.402873926 | down-regulated |
| O75636 | FCN3 | 0.411921293 | down-regulated |
| P01008 | SERPINC1 | 0.415646679 | down-regulated |
| Q961Y4 | CPB2 | 0.418839868 | down-regulated |
| P01602 | IGKV1-5 | 0.426822839 | down-regulated |
| P02753 | RBP4 | 0.430548225 | down-regulated |
| P06727 | APOA4 | 0.432677017 | down-regulated |
| Q96PD5 | PGLYRP2 | 0.434805809 | down-regulated |
| P01768 | IGHV3-30 | 0.450239553 | down-regulated |
| P02768 | ALB | 0.455561533 | down-regulated |
| P20851 | C4BPB | 0.469930881 | down-regulated |
| P43652 | AFM | 0.470463079 | down-regulated |
| P02774 | GC | 0.477381654 | down-regulated |
| P01861 | IGHG4 | 0.480574842 | down-regulated |
| P69905 | HBA2 | 0.48376803 | down-regulated |
| P08185 | SERPINA6 | 0.486961219 | down-regulated |
| P08697 | SERPINF2 | 0.492815397 | down-regulated |
| P10909 | CLU | 0.496008585 | down-regulated |
| Q08380 | LGALS3BP | 0.49760518 | down-regulated |
| P03952 | KLKB1 | 0.498137378 | down-regulated |

TABLE 4
Differential Expression Analysis of Plasma Proteins in Pancreatic
Cancer Patients Compared to Healthy Controls.

| | Gene | Normalized | |
|---|---|---|---|
| Accession | name | ratio | Trend |
| P10643 | C7 | 2.279126038 | up-regulated |
| P02748 | C9 | 2.167857162 | up-regulated |
| P05160 | F13B | 3.339952183 | up-regulated |
| P27824 | CANX | 2.105622028 | up-regulated |
| Q14697 | GANAB | 2.314958388 | up-regulated |
| P50148 | GNAQ | 2.159370553 | up-regulated |
| P0DJI8 | SAA1 | 2.333817519 | up-regulated |
| P02741 | CRP | 6.163164164 | up-regulated |
| P0DJI9 | SAA2 | 94.29565734 | up-regulated |
| O43294 | TGFB1I1 | 94.29565734 | up-regulated |
| P06744 | GPI | 94.29565734 | up-regulated |
| O15143 | ARPC1B | 94.29565734 | up-regulated |
| P55209 | NAP1L1 | 94.29565734 | up-regulated |
| Q9Y251 | HPSE | 94.29565734 | up-regulated |
| Q8N0Y7 | PGAM4 | 94.29565734 | up-regulated |
| P55058 | PLTP | 94.29565734 | up-regulated |
| A0A0A0MT36 | IGKV6D-21 | 0.009429566 | down-regulated |
| P02655 | APOC2 | 0.297974277 | down-regulated |
| P35858 | IGFALS | 0.410186109 | down-regulated |
| P02656 | APOC3 | 0.346065062 | down-regulated |
| P04114 | APOB | 0.395098804 | down-regulated |
| P02652 | APOA2 | 0.303632017 | down-regulated |
| P27169 | PON1 | 0.44036072 | down-regulated |
| P02647 | APOA1 | 0.479021939 | down-regulated |
| P05452 | CLEC3B | 0.449790286 | down-regulated |
| P04278 | SHBG | 0.359266454 | down-regulated |
| P05154 | SERPINA5 | 0.388498108 | down-regulated |
| O75636 | FCN3 | 0.291373581 | down-regulated |
| Q96IY4 | CPB2 | 0.009429566 | down-regulated |
| P01768 | IGHV3-30 | 0.093352701 | down-regulated |
| Q08380 | LGALS3BP | 0.466763504 | down-regulated |
| P0DOX5 | Immuno- | 0.454505068 | down-regulated |
| | globulin | | |
| | gamma-1 | | |
| | heavy | | |
| | chain | | |
| P01597 | IGKV1-39 | 0.358323498 | down-regulated |
| P10720 | PF4V1 | 0.361152368 | down-regulated |
| P01593 | IGKV1D-33 | 0.488451505 | down-regulated |
| P06310 | IGKV2-30 | 0.354551672 | down-regulated |
| P62873 | GNB1 | 0.009429566 | down-regulated |
| P31946 | YWHAB | 0.009429566 | down-regulated |
| Q9H0U4 | RAB1B | 0.009429566 | down-regulated |
| Q9UK55 | SERPINA10 | 0.497881071 | down-regulated |
| P10809 | HSPD1 | 0.009429566 | down-regulated |
| P10321 | HLA-C | 0.468649417 | down-regulated |
| P15169 | CPN1 | 0.497881071 | down-regulated |
| A0A075B6R2 | IGHV4-4 | 0.009429566 | down-regulated |
| P04632 | CAPNS1 | 0.009429566 | down-regulated |
| P01889 | HLA-B | 0.207450446 | down-regulated |
| P09493 | TPM1 | 0.009429566 | down-regulated |
| A0A0C4DH29 | IGHV1-3 | 0.009429566 | down-regulated |
| Q71U36 | TUBA1A | 0.4733642 | down-regulated |
| P01137 | TGFB1 | 0.009429566 | down-regulated |

TABLE 5
Differential Expression Analysis of Plasma Proteins
in DLB Patient Compared to Healthy Controls.

| | Gene | Normalized | |
|---|---|---|---|
| Accession | name | ratio | Trend |
| P01861 | IGHG4 | 2.517046607 | Up-regulated |
| A0A0J9YXX1 | IGHV5-10-1 | 2.44438575 | Up-regulated |
| P01876 | IGHA1 | 2.301858685 | Up-regulated |
| P0DOX2 | Immuno- | 2.476058431 | Up-regulated |
| | globulin | | |
| | alpha-2 | | |
| | heavy | | |
| | chain | | |
| P02751 | FN1 | 2.672615365 | Up-regulated |
| P18428 | LBP | 3.002383869 | Up-regulated |
| A0A0C4DH34 | IGHV4-28 | 2.295337838 | Up-regulated |
| P02760 | AMBP | 3.375003648 | Up-regulated |
| P01763 | IGHV3-48 | 93.15494474 | Up-regulated |
| P04439 | HLA-A | 93.15494474 | Up-regulated |
| P00742 | F10 | 93.15494474 | Up-regulated |
| A0A075B6S5 | IGKV1-27 | 93.15494474 | Up-regulated |
| Q9UK55 | SERPINA10 | 0.365167383 | Down-regulated |
| Q9HBI1 | PARVB | 9.32E−03 | Down-regulated |
| P0DOY2 | IGLC2 | 0.436896691 | Down-regulated |
| P02766 | TTR | 0.292506526 | Down-regulated |
| P69905 | HBA1 | 0.425718097 | Down-regulated |
| P68871 | HBB | 0.359578087 | Down-regulated |
| P05452 | CLEC3B | 9.32E−03 | Down-regulated |
| P00915 | CA1 | 0.466706273 | Down-regulated |
| P02647 | APOA1 | 0.418265702 | Down-regulated |
| P27105 | STOM | 9.32E−03 | Down-regulated |
| P02745 | C1QA | 9.32E−03 | Down-regulated |
| O43866 | CD5L | 0.363304284 | Down-regulated |
| P40197 | GP5 | 9.32E−03 | Down-regulated |
| P35858 | IGFALS | 0.439691339 | Down-regulated |
| P15169 | CPN1 | 0.251518351 | Down-regulated |
| A0A0C4DH69 | IGKV1-9 | 9.32E−03 | Down-regulated |
| P00488 | F13A1 | 9.32E−03 | Down-regulated |
| P01871 | IGHM | 0.354920339 | Down-regulated |
| P02652 | APOA2 | 9.32E−03 | Down-regulated |
| P35527 | KRT9 | 9.32E−03 | Down-regulated |
| P05543 | SERPINA7 | 0.48906346 | Down-regulated |
| P62258 | YWHAE | 9.32E−03 | Down-regulated |

TABLE 6
Differential Expression Analysis of Plasma Proteins
in AD Patient Compared to Healthy Controls.

| | Gene | Normalized | |
|---|---|---|---|
| Accession | name | ratio | Trend |
| P02751 | FN1 | 15.44026846 | up-regulated |
| Q8N2S1 | LTBP4 | 18.31006711 | up-regulated |
| O15031 | PLXNB2 | 134.2281879 | up-regulated |
| Q8TER0 | SNED1 | 134.2281879 | up-regulated |
| P43652 | AFM | 134.2281879 | up-regulated |
| P16157 | ANK1 | 134.2281879 | up-regulated |
| P11277 | SPTB | 134.2281879 | up-regulated |
| Q92876 | KLK6 | 134.2281879 | up-regulated |
| P35442 | THBS2 | 134.2281879 | up-regulated |
| Q562R1 | ACTBL2 | 134.2281879 | up-regulated |
| O14791 | APOL1 | 134.2281879 | up-regulated |
| P05121 | SERPINE1 | 134.2281879 | up-regulated |
| P07355 | ANXA2 | 134.2281879 | up-regulated |
| P23515 | OMG | 134.2281879 | up-regulated |
| P12814 | ACTN1 | 57.99194631 | up-regulated |
| O43866 | CD5L | 134.2281879 | up-regulated |
| P80108 | GPLD1 | 134.2281879 | up-regulated |
| Q92823 | NRCAM | 134.2281879 | up-regulated |
| Q14767 | LTBP2 | 134.2281879 | up-regulated |
| Q14520 | HABP2 | 134.2281879 | up-regulated |
| P21810 | BGN | 20.18657718 | up-regulated |
| P43251 | BTD | 134.2281879 | up-regulated |
| P04114 | APOB | 134.2281879 | up-regulated |
| Q9H2X0 | CHRD | 134.2281879 | up-regulated |
| A0A087WSY6 | IGKV3D-15 | 134.2281879 | up-regulated |
| P20851 | C4BPB | 134.2281879 | up-regulated |
| A0A0J9YXX1 | IGHV5-10-1 | 134.2281879 | up-regulated |
| P02042 | HBD | 134.2281879 | up-regulated |
| Q6YHK3 | CD109 | 134.2281879 | up-regulated |
| Q6ZRP7 | QSOX2 | 134.2281879 | up-regulated |
| Q02809 | PLOD1 | 134.2281879 | up-regulated |
| Q96KG7 | MEGF10 | 134.2281879 | up-regulated |
| P11362 | FGFR1 | 134.2281879 | up-regulated |
| P49257 | LMAN1 | 134.2281879 | up-regulated |
| P00915 | CA1 | 134.2281879 | up-regulated |
| Q9C0A0 | CNTNAP4 | 134.2281879 | up-regulated |
| P49641 | MAN2A2 | 134.2281879 | up-regulated |
| Q8IV08 | PLD3 | 134.2281879 | up-regulated |
| Q9UBG0 | MRC2 | 134.2281879 | up-regulated |
| P02533 | KRT14 | 134.2281879 | up-regulated |
| P35052 | GPC1 | 134.2281879 | up-regulated |
| P60033 | CD81 | 26.24026846 | up-regulated |
| P27105 | STOM | 134.2281879 | up-regulated |
| P12277 | CKB | 134.2281879 | up-regulated |
| O14594 | NCAN | 134.2281879 | up-regulated |
| Q99972 | MYOC | 134.2281879 | up-regulated |
| Q9NPR2 | SEMA4B | 134.2281879 | up-regulated |
| P47972 | NPTX2 | 134.2281879 | up-regulated |
| P02461 | COL3A1 | 134.2281879 | up-regulated |
| Q8IXL6 | FAM20C | 134.2281879 | up-regulated |
| P07942 | LAMB1 | 134.2281879 | up-regulated |
| Q06033 | ITIH3 | 134.2281879 | up-regulated |
| Q7Z3B1 | NEGR1 | 134.2281879 | up-regulated |
| P01602 | IGKV1-5 | 16.91946309 | up-regulated |
| P11047 | LAMC1 | 134.2281879 | up-regulated |
| Q53RD9 | FBLN7 | 134.2281879 | up-regulated |
| P12110 | COL6A2 | 134.2281879 | up-regulated |
| P02549 | SPTA1 | 134.2281879 | up-regulated |
| Q6UX72 | B3GNT9 | 134.2281879 | up-regulated |
| Q02985 | CFHR3 | 134.2281879 | up-regulated |
| P43234 | CTSO | 134.2281879 | up-regulated |
| Q14766 | LTBP1 | 134.2281879 | up-regulated |
| Q9NT99 | LRRC4B | 134.2281879 | up-regulated |
| Q96FE5 | LINGO1 | 134.2281879 | up-regulated |
| Q9ULB1 | NRXN1 | 134.2281879 | up-regulated |
| P23468 | PTPRD | 134.2281879 | up-regulated |
| P04070 | PROC | 134.2281879 | up-regulated |
| P08779 | KRT16 | 134.2281879 | up-regulated |
| Q6UWH4 | GASK1B | 134.2281879 | up-regulated |
| Q13751 | LAMB3 | 134.2281879 | up-regulated |
| A0A0C4DH38 | IGHV5-51 | 134.2281879 | up-regulated |
| Q8N475 | FSTL5 | 134.2281879 | up-regulated |
| Q96PX8 | SLITRK1 | 134.2281879 | up-regulated |
| Q9Y6N7 | ROBO1 | 134.2281879 | up-regulated |
| P48740 | MASP1 | 134.2281879 | up-regulated |
| P22105 | TNXB | 134.2281879 | up-regulated |
| P32004 | L1CAM | 134.2281879 | up-regulated |
| Q6UXD5 | SEZ6L2 | 134.2281879 | up-regulated |
| Q8N436 | CPXM2 | 134.2281879 | up-regulated |
| P00749 | PLAU | 134.2281879 | up-regulated |
| Q92752 | TNR | 134.2281879 | up-regulated |
| Q9HDB5 | NRXN3 | 134.2281879 | up-regulated |
| Q99536 | VAT1 | 134.2281879 | up-regulated |
| Q7Z7M0 | MEGF8 | 134.2281879 | up-regulated |
| P22004 | BMP6 | 134.2281879 | up-regulated |
| Q86YZ3 | HRNR | 134.2281879 | up-regulated |
| Q9UM47 | NOTCH3 | 134.2281879 | up-regulated |
| Q24JP5 | TMEM132A | 134.2281879 | up-regulated |
| P62805 | H4C1 | 0.013422819 | down-regulated |
| P09651 | HNRNPA1 | 0.013422819 | down-regulated |
| P63104 | YWHAZ | 0.017449664 | down-regulated |
| P22626 | HNRNPA2B1 | 0.013422819 | down-regulated |
| P51991 | HNRNPA3 | 0.017449664 | down-regulated |
| Q99729 | HNRNPAB | 0.013422819 | down-regulated |
| P08865 | RPSA | 0.013422819 | down-regulated |
| P40925 | MDH1 | 0.013422819 | down-regulated |
| Q10567 | AP1B1 | 0.013422819 | down-regulated |
| P62318 | SNRPD3 | 0.013422819 | down-regulated |
| P30050 | RPL12 | 0.013422819 | down-regulated |
| P52907 | CAPZA1 | 0.013422819 | down-regulated |
| P21281 | ATP6V1B2 | 0.013422819 | down-regulated |
| Q9Y2X3 | NOP58 | 0.013422819 | down-regulated |
| Q13148 | TARDBP | 0.013422819 | down-regulated |
| P50991 | CCT4 | 0.013422819 | down-regulated |
| Q14195 | DPYSL3 | 0.013422819 | down-regulated |
| P47756 | CAPZB | 0.013422819 | down-regulated |
| P26641 | EEF1G | 0.013422819 | down-regulated |
| O15145 | ARPC3 | 0.013422819 | down-regulated |
| Q14974 | KPNB1 | 0.013422819 | down-regulated |
| Q92520 | FAM3C | 0.013422819 | down-regulated |
| P63244 | RACK1 | 0.013422819 | down-regulated |
| Q13509 | TUBB3 | 0.013422819 | down-regulated |
| P37802 | TAGLN2 | 0.013422819 | down-regulated |
| P27348 | YWHAQ | 0.013422819 | down-regulated |
| P52272 | HNRNPM | 0.013422819 | down-regulated |
| Q99832 | CCT7 | 0.013422819 | down-regulated |
| P08621 | SNRNP70 | 0.013422819 | down-regulated |
| P18669 | PGAM1 | 0.013422819 | down-regulated |
| Q1KMD3 | HNRNPUL2 | 0.013422819 | down-regulated |
| P63261 | ACTG1 | 0.013422819 | down-regulated |
| P49368 | CCT3 | 0.013422819 | down-regulated |
| P41250 | GARS1 | 0.013422819 | down-regulated |
| P11940 | PABPC1 | 0.013422819 | down-regulated |
| Q7KZF4 | SND1 | 0.013422819 | down-regulated |
| Q08211 | DHX9 | 0.013422819 | down-regulated |
| P08758 | ANXA5 | 0.013422819 | down-regulated |
| Q15029 | EFTUD2 | 0.013422819 | down-regulated |
| Q12906 | ILF3 | 0.013422819 | down-regulated |
| Q13838 | DDX39B | 0.013422819 | down-regulated |
| P50395 | GDI2 | 0.013422819 | down-regulated |
| Q15063 | POSTN | 0.013422819 | down-regulated |
| P61026 | RAB10 | 0.013422819 | down-regulated |
| P68371 | TUBB4B | 0.013422819 | down-regulated |
| Q01105 | SET | 0.013422819 | down-regulated |
| P05388 | RPLP0 | 0.013422819 | down-regulated |
| P55795 | HNRNPH2 | 0.013422819 | down-regulated |
| Q8TAG5 | VSTM2A | 0.013422819 | down-regulated |
| P22087 | FBL | 0.013422819 | down-regulated |
| P62995 | TRA2B | 0.013422819 | down-regulated |
| Q86VP6 | CAND1 | 0.013422819 | down-regulated |
| P04908 | H2AC4 | 0.013422819 | down-regulated |
| P07437 | TUBB | 0.013422819 | down-regulated |
| Q96DA2 | RAB39B | 0.013422819 | down-regulated |
| P06753 | TPM3 | 0.013422819 | down-regulated |
| Q96IY4 | CPB2 | 0.013422819 | down-regulated |
| P62826 | RAN | 0.013422819 | down-regulated |
| P84243 | H3-3A | 0.013422819 | down-regulated |
| P55072 | VCP | 0.013422819 | down-regulated |
| Q15717 | ELAVL1 | 0.013422819 | down-regulated |
| Q9BQE3 | TUBA1C | 0.013422819 | down-regulated |
| O60814 | H2BC12 | 0.013422819 | down-regulated |
| P10809 | HSPD1 | 0.013422819 | down-regulated |
| Q15185 | PTGES3 | 0.013422819 | down-regulated |
| P62873 | GNB1 | 0.013422819 | down-regulated |
| P07814 | EPRS | 0.013422819 | down-regulated |
| A0A0C4DH69 | IGKV1-9 | 0.013422819 | down-regulated |
| P61224 | RAP1B | 0.013422819 | down-regulated |
| Q9ULV4 | CORO1C | 0.013422819 | down-regulated |
| Q8N2Q7 | NLGN1 | 0.013422819 | down-regulated |
| P45973 | CBX5 | 0.013422819 | down-regulated |
| Q9HCJ6 | VAT1L | 0.013422819 | down-regulated |
| P62424 | RPL7A | 0.013422819 | down-regulated |
| P26599 | PTBP1 | 0.013422819 | down-regulated |
| P26378 | ELAVL4 | 0.013422819 | down-regulated |
| P29401 | TKT | 0.013422819 | down-regulated |
| P61978 | HNRNPK | 0.013422819 | down-regulated |
| P54136 | RARS | 0.013422819 | down-regulated |
| P05141 | SLC25A5 | 0.013422819 | down-regulated |
| P57721 | PCBP3 | 0.013422819 | down-regulated |
| P18621 | RPL17 | 0.013422819 | down-regulated |
| P40926 | MDH2 | 0.013422819 | down-regulated |
| P53396 | ACLY | 0.013422819 | down-regulated |
| P63241 | EIF5A | 0.013422819 | down-regulated |
| P60842 | EIF4A1 | 0.013422819 | down-regulated |
| A0M8Q6 | IGLC7 | 0.013422819 | down-regulated |
| Q9Y265 | RUVBL1 | 0.013422819 | down-regulated |
| Q09328 | MGAT5 | 0.013422819 | down-regulated |
| P15311 | EZR | 0.013422819 | down-regulated |
| O60506 | SYNCRIP | 0.013422819 | down-regulated |
| P17174 | GOT1 | 0.013422819 | down-regulated |
| Q71U36 | TUBA1A | 0.013422819 | down-regulated |
| P68104 | EEF1A1 | 0.013422819 | down-regulated |
| Q15366 | PCBP2 | 0.013422819 | down-regulated |
| Q00839 | HNRNPU | 0.013422819 | down-regulated |
| O14980 | XPO1 | 0.013422819 | down-regulated |
| P50990 | CCT8 | 0.013422819 | down-regulated |
| P31943 | HNRNPH1 | 0.013422819 | down-regulated |
| P21796 | VDAC1 | 0.013422819 | down-regulated |
| P52597 | HNRNPF | 0.013422819 | down-regulated |
| Q08945 | SSRP1 | 0.013422819 | down-regulated |
| P12235 | SLC25A4 | 0.013422819 | down-regulated |
| P00558 | PGK1 | 0.013422819 | down-regulated |
| P35637 | FUS | 0.013422819 | down-regulated |
| P67809 | YBX1 | 0.013422819 | down-regulated |
| Q02543 | RPL18A | 0.013422819 | down-regulated |
| P26639 | TARS1 | 0.013422819 | down-regulated |
| P61981 | YWHAG | 0.013422819 | down-regulated |
| Q12905 | ILF2 | 0.013422819 | down-regulated |
| Q16643 | DBN1 | 0.013422819 | down-regulated |
| P07910 | HNRNPC | 0.013422819 | down-regulated |
| P62913 | RPL11 | 0.013422819 | down-regulated |
| P23396 | RPS3 | 0.013422819 | down-regulated |
| P62937 | PPIA | 0.013422819 | down-regulated |
| P06576 | ATP5F1B | 0.013422819 | down-regulated |
| P68431 | H3C1 | 0.013422819 | down-regulated |
| Q14103 | HNRNPD | 0.013422819 | down-regulated |
| P55209 | NAP1L1 | 0.013422819 | down-regulated |
| P63010 | AP2B1 | 0.013422819 | down-regulated |
| P46781 | RPS9 | 0.013422819 | down-regulated |
| O14979 | HNRNPDL | 0.013422819 | down-regulated |
| P62241 | RPS8 | 0.013422819 | down-regulated |
| Q9Y3U8 | RPL36 | 0.013422819 | down-regulated |
| P68363 | TUBA1B | 0.013422819 | down-regulated |
| P54577 | YARS | 0.013422819 | down-regulated |
| P35580 | MYH10 | 0.013422819 | down-regulated |
| P38159 | RBMX | 0.013422819 | down-regulated |
| P41219 | PRPH | 0.013422819 | down-regulated |
| Q8TC07 | TBC1D15 | 0.013422819 | down-regulated |
| Q00610 | CLTC | 0.013422819 | down-regulated |
| Q8IZA0 | KIAA0319L | 0.013422819 | down-regulated |
| P19022 | CDH2 | 0.013422819 | down-regulated |
| P04350 | TUBB4A | 0.013422819 | down-regulated |
| Q16629 | SRSF7 | 0.013422819 | down-regulated |
| Q6FHJ7 | SFRP4 | 0.013422819 | down-regulated |
| P84103 | SRSF3 | 0.013422819 | down-regulated |
| P61313 | RPL15 | 0.013422819 | down-regulated |
| P46783 | RPS10 | 0.013422819 | down-regulated |
| P18124 | RPL7 | 0.013422819 | down-regulated |
| P17844 | DDX5 | 0.013422819 | down-regulated |
| Q14194 | CRMP1 | 0.013422819 | down-regulated |
| Q71DI3 | HIST2H3A | 0.013422819 | down-regulated |
| P0DN76 | U2AF1L5 | 0.013422819 | down-regulated |
| P61247 | RPS3A | 0.013422819 | down-regulated |
| Q9UQ80 | PA2G4 | 0.013422819 | down-regulated |
| P16402 | H1-3 | 0.013422819 | down-regulated |
| Q99623 | PHB2 | 0.013422819 | down-regulated |
| P07237 | P4HB | 0.013422819 | down-regulated |
| P14866 | HNRNPL | 0.013422819 | down-regulated |
| O75367 | H2AFY | 0.013422819 | down-regulated |
| P27635 | RPL10 | 0.013422819 | down-regulated |
| O75533 | SF3B1 | 0.013422819 | down-regulated |
| P78371 | CCT2 | 0.013422819 | down-regulated |
| P0DP23 | CALM1 | 0.013422819 | down-regulated |
| P46777 | RPL5 | 0.013422819 | down-regulated |
| P16104 | H2AFX | 0.013422819 | down-regulated |
| P62701 | RPS4X | 0.013422819 | down-regulated |
| P40227 | CCT6A | 0.013422819 | down-regulated |
| Q5EB52 | MEST | 0.013422819 | down-regulated |
| P83731 | RPL24 | 0.013422819 | down-regulated |
| P55884 | EIF3B | 0.013422819 | down-regulated |
| P49588 | AARS | 0.013422819 | down-regulated |
| Q5JXB2 | UBE2NL | 0.013422819 | down-regulated |
| P19338 | NCL | 0.013422819 | down-regulated |
| P17987 | TCP1 | 0.013422819 | down-regulated |
| P62847 | RPS24 | 0.013422819 | down-regulated |
| P60660 | MYL6 | 0.013422819 | down-regulated |
| P11413 | G6PD | 0.013422819 | down-regulated |
| P62081 | RPS7 | 0.013422819 | down-regulated |

TABLE 7
Differential Expression Analysis of Plasma Proteins
in PD Patient Compared to Healthy Controls.

| | Gene | Normalized | |
|---|---|---|---|
| Accession | name | ratio | Trend |
| P02751 | FN1 | 13.67028494 | up-regulated |
| P01023 | A2M | 14.52917232 | up-regulated |
| P00450 | CP | 15.68928087 | up-regulated |
| Q14515 | SPARCL1 | 23.24694708 | up-regulated |
| O00533 | CHL1 | 31.20624152 | up-regulated |
| P78509 | RELN | 18.90502035 | up-regulated |
| Q8N2S1 | LTBP4 | 14.40976934 | up-regulated |
| O15031 | PLXNB2 | 135.6852103 | up-regulated |
| Q8TER0 | SNED1 | 135.6852103 | up-regulated |
| P05060 | CHGB | 13.85345997 | up-regulated |
| P43652 | AFM | 135.6852103 | up-regulated |
| P16157 | ANK1 | 135.6852103 | up-regulated |
| Q92876 | KLK6 | 135.6852103 | up-regulated |
| P35442 | THBS2 | 135.6852103 | up-regulated |
| Q562R1 | ACTBL2 | 135.6852103 | up-regulated |
| Q53EL9 | SEZ6 | 24.49253731 | up-regulated |
| O14791 | APOL1 | 135.6852103 | up-regulated |
| P05121 | SERPINE1 | 135.6852103 | up-regulated |
| P07355 | ANXA2 | 135.6852103 | up-regulated |
| P23515 | OMG | 135.6852103 | up-regulated |
| Q06481 | APLP2 | 34.34871099 | up-regulated |
| P80108 | GPLD1 | 135.6852103 | up-regulated |
| Q92823 | NRCAM | 135.6852103 | up-regulated |
| Q14767 | LTBP2 | 135.6852103 | up-regulated |
| Q14520 | HABP2 | 135.6852103 | up-regulated |
| Q99435 | NELL2 | 14.16824966 | up-regulated |
| P21810 | BGN | 17.48032564 | up-regulated |
| P43251 | BTD | 135.6852103 | up-regulated |
| Q9H2X0 | CHRD | 135.6852103 | up-regulated |
| A0A087WSY6 | IGKV3D-15 | 135.6852103 | up-regulated |
| A0A0J9YXX1 | IGHV5-10-1 | 135.6852103 | up-regulated |
| Q6YHK3 | CD109 | 135.6852103 | up-regulated |
| Q6ZRP7 | QSOX2 | 135.6852103 | up-regulated |
| Q02809 | PLOD1 | 135.6852103 | up-regulated |
| Q96KG7 | MEGF10 | 135.6852103 | up-regulated |
| P11362 | FGFR1 | 135.6852103 | up-regulated |
| P49257 | LMAN1 | 135.6852103 | up-regulated |
| Q9C0A0 | CNTNAP4 | 135.6852103 | up-regulated |
| P49641 | MAN2A2 | 135.6852103 | up-regulated |
| Q8IV08 | PLD3 | 135.6852103 | up-regulated |
| Q9UBG0 | MRC2 | 135.6852103 | up-regulated |
| P02533 | KRT14 | 135.6852103 | up-regulated |
| P35052 | GPC1 | 135.6852103 | up-regulated |
| P12277 | CKB | 135.6852103 | up-regulated |
| O14594 | NCAN | 135.6852103 | up-regulated |
| Q99972 | MYOC | 135.6852103 | up-regulated |
| Q9NPR2 | SEMA4B | 135.6852103 | up-regulated |
| P47972 | NPTX2 | 135.6852103 | up-regulated |
| P02461 | COL3A1 | 135.6852103 | up-regulated |
| Q81XL6 | FAM20C | 135.6852103 | up-regulated |
| P07942 | LAMB1 | 135.6852103 | up-regulated |
| Q9BYH1 | SEZ6L | 15.35142469 | up-regulated |
| Q7Z3B1 | NEGR1 | 135.6852103 | up-regulated |
| P11047 | LAMC1 | 135.6852103 | up-regulated |
| Q53RD9 | FBLN7 | 135.6852103 | up-regulated |
| P12110 | COL6A2 | 135.6852103 | up-regulated |
| Q6UX72 | B3GNT9 | 135.6852103 | up-regulated |
| Q02985 | CFHR3 | 135.6852103 | up-regulated |
| P43234 | CTSO | 135.6852103 | up-regulated |
| Q14766 | LTBP1 | 135.6852103 | up-regulated |
| Q9NT99 | LRRC4B | 135.6852103 | up-regulated |
| Q96FE5 | LINGO1 | 135.6852103 | up-regulated |
| Q9ULB1 | NRXN1 | 135.6852103 | up-regulated |
| P23468 | PTPRD | 135.6852103 | up-regulated |
| P04070 | PROC | 135.6852103 | up-regulated |
| P08779 | KRT16 | 135.6852103 | up-regulated |
| A0A0C4DH38 | IGHV5-51 | 135.6852103 | up-regulated |
| Q96PX8 | SLITRK1 | 135.6852103 | up-regulated |
| Q9Y6N7 | ROBO1 | 135.6852103 | up-regulated |
| P48740 | MASP1 | 135.6852103 | up-regulated |
| P32004 | L1CAM | 135.6852103 | up-regulated |
| Q6UXD5 | SEZ6L2 | 135.6852103 | up-regulated |
| Q8N436 | CPXM2 | 135.6852103 | up-regulated |
| P00749 | PLAU | 135.6852103 | up-regulated |
| Q92752 | TNR | 135.6852103 | up-regulated |
| Q9HDB5 | NRXN3 | 135.6852103 | up-regulated |
| Q99536 | VAT1 | 135.6852103 | up-regulated |
| Q7Z7M0 | MEGF8 | 135.6852103 | up-regulated |
| P22004 | BMP6 | 135.6852103 | up-regulated |
| Q86YZ3 | HRNR | 135.6852103 | up-regulated |
| Q9UM47 | NOTCH3 | 135.6852103 | up-regulated |
| Q24JP5 | TMEM132A | 135.6852103 | up-regulated |
| P10586 | PTPRF | 135.6852103 | up-regulated |
| P55268 | LAMB2 | 135.6852103 | up-regulated |
| Q16706 | MAN2A1 | 135.6852103 | up-regulated |
| P12814 | ACTN1 | 0.013568521 | down-regulated |
| P00739 | HPR | 0.013568521 | down-regulated |
| P01782 | IGHV3-9 | 0.013568521 | down-regulated |
| O43143 | DHX15 | 0.013568521 | down-regulated |
| P60900 | PSMA6 | 0.013568521 | down-regulated |
| P50502 | ST13 | 0.013568521 | down-regulated |
| P23528 | CFL1 | 0.013568521 | down-regulated |
| P00338 | LDHA | 0.013568521 | down-regulated |
| P62805 | H4C1 | 0.013568521 | down-regulated |
| P11142 | HSPA8 | 0.013568521 | down-regulated |
| P68400 | CSNK2A1 | 0.013568521 | down-regulated |
| P36578 | RPL4 | 0.013568521 | down-regulated |
| P60174 | TPI1 | 0.013568521 | down-regulated |
| P04259 | KRT6B | 0.013568521 | down-regulated |
| P50914 | RPL14 | 0.013568521 | down-regulated |
| P22626 | HNRNPA2B1 | 0.013568521 | down-regulated |
| O00567 | NOP56 | 0.013568521 | down-regulated |
| P51991 | HNRNPA3 | 0.013568521 | down-regulated |
| P13639 | EEF2 | 0.013568521 | down-regulated |
| Q99729 | HNRNPAB | 0.013568521 | down-regulated |
| P08865 | RPSA | 0.013568521 | down-regulated |
| P40925 | MDH1 | 0.013568521 | down-regulated |
| Q10567 | AP1B1 | 0.013568521 | down-regulated |
| P62318 | SNRPD3 | 0.013568521 | down-regulated |
| P30050 | RPL12 | 0.013568521 | down-regulated |
| P52907 | CAPZA1 | 0.013568521 | down-regulated |
| P21281 | ATP6V1B2 | 0.013568521 | down-regulated |
| Q9Y2X3 | NOP58 | 0.013568521 | down-regulated |
| Q13148 | TARDBP | 0.013568521 | down-regulated |
| P50991 | CCT4 | 0.013568521 | down-regulated |
| Q14195 | DPYSL3 | 0.013568521 | down-regulated |
| P26641 | EEF1G | 0.013568521 | down-regulated |
| O15145 | ARPC3 | 0.013568521 | down-regulated |
| Q14974 | KPNB1 | 0.013568521 | down-regulated |
| Q92520 | FAM3C | 0.013568521 | down-regulated |
| Q13509 | TUBB3 | 0.013568521 | down-regulated |
| P37802 | TAGLN2 | 0.013568521 | down-regulated |
| P27348 | YWHAQ | 0.013568521 | down-regulated |
| P52272 | HNRNPM | 0.013568521 | down-regulated |
| Q99832 | CCT7 | 0.013568521 | down-regulated |
| P08621 | SNRNP70 | 0.013568521 | down-regulated |
| P18669 | PGAM1 | 0.013568521 | down-regulated |
| Q1KMD3 | HNRNPUL2 | 0.013568521 | down-regulated |
| P63261 | ACTG1 | 0.013568521 | down-regulated |
| P49368 | CCT3 | 0.013568521 | down-regulated |
| P41250 | GARS1 | 0.013568521 | down-regulated |
| Q7KZF4 | SND1 | 0.013568521 | down-regulated |
| Q08211 | DHX9 | 0.013568521 | down-regulated |
| P08758 | ANXA5 | 0.013568521 | down-regulated |
| Q15029 | EFTUD2 | 0.013568521 | down-regulated |
| Q12906 | ILF3 | 0.013568521 | down-regulated |
| Q13838 | DDX39B | 0.013568521 | down-regulated |
| P50395 | GDI2 | 0.013568521 | down-regulated |
| Q15063 | POSTN | 0.013568521 | down-regulated |
| P61026 | RAB10 | 0.013568521 | down-regulated |
| P68371 | TUBB4B | 0.013568521 | down-regulated |
| Q01105 | SET | 0.013568521 | down-regulated |
| P05388 | RPLP0 | 0.013568521 | down-regulated |
| P55795 | HNRNPH2 | 0.013568521 | down-regulated |
| P22087 | FBL | 0.013568521 | down-regulated |
| P62995 | TRA2B | 0.013568521 | down-regulated |
| Q86VP6 | CAND1 | 0.013568521 | down-regulated |
| P04908 | H2AC4 | 0.013568521 | down-regulated |
| P07437 | TUBB | 0.013568521 | down-regulated |
| Q96DA2 | RAB39B | 0.013568521 | down-regulated |
| P06753 | TPM3 | 0.013568521 | down-regulated |
| Q96IY4 | CPB2 | 0.013568521 | down-regulated |
| P62826 | RAN | 0.013568521 | down-regulated |
| P84243 | H3-3A | 0.013568521 | down-regulated |
| P55072 | VCP | 0.013568521 | down-regulated |
| Q15717 | ELAVL1 | 0.013568521 | down-regulated |
| Q9BQE3 | TUBA1C | 0.013568521 | down-regulated |
| O60814 | H2BC12 | 0.013568521 | down-regulated |
| Q15185 | PTGES3 | 0.013568521 | down-regulated |
| P62873 | GNB1 | 0.013568521 | down-regulated |
| P07814 | EPRS | 0.013568521 | down-regulated |
| A0A0C4DH69 | IGKV1-9 | 0.013568521 | down-regulated |
| P61224 | RAP1B | 0.013568521 | down-regulated |
| Q9ULV4 | CORO1C | 0.013568521 | down-regulated |
| Q8N2Q7 | NLGN1 | 0.013568521 | down-regulated |
| P45973 | CBX5 | 0.013568521 | down-regulated |
| Q9HCJ6 | VAT1L | 0.013568521 | down-regulated |
| P62424 | RPL7A | 0.013568521 | down-regulated |
| P26599 | PTBP1 | 0.013568521 | down-regulated |
| P29401 | TKT | 0.013568521 | down-regulated |
| P61978 | HNRNPK | 0.013568521 | down-regulated |
| P54136 | RARS | 0.013568521 | down-regulated |
| P05141 | SLC25A5 | 0.013568521 | down-regulated |
| P57721 | PCBP3 | 0.013568521 | down-regulated |
| P18621 | RPL17 | 0.013568521 | down-regulated |
| P40926 | MDH2 | 0.013568521 | down-regulated |
| P53396 | ACLY | 0.013568521 | down-regulated |
| P63241 | EIF5A | 0.013568521 | down-regulated |
| P60842 | EIF4A1 | 0.013568521 | down-regulated |
| A0M8Q6 | IGLC7 | 0.013568521 | down-regulated |
| Q9Y265 | RUVBL1 | 0.013568521 | down-regulated |
| Q09328 | MGAT5 | 0.013568521 | down-regulated |
| P15311 | EZR | 0.013568521 | down-regulated |
| O60506 | SYNCRIP | 0.013568521 | down-regulated |
| P17174 | GOT1 | 0.013568521 | down-regulated |
| Q71U36 | TUBA1A | 0.013568521 | down-regulated |
| P68104 | EEF1A1 | 0.013568521 | down-regulated |
| Q15366 | PCBP2 | 0.013568521 | down-regulated |
| O14980 | XPO1 | 0.013568521 | down-regulated |
| P31943 | HNRNPH1 | 0.013568521 | down-regulated |
| P21796 | VDAC1 | 0.013568521 | down-regulated |
| P52597 | HNRNPF | 0.013568521 | down-regulated |
| P12235 | SLC25A4 | 0.013568521 | down-regulated |
| P00558 | PGK1 | 0.013568521 | down-regulated |
| P35637 | FUS | 0.013568521 | down-regulated |
| P67809 | YBX1 | 0.013568521 | down-regulated |
| Q02543 | RPL18A | 0.013568521 | down-regulated |
| P26639 | TARS1 | 0.013568521 | down-regulated |
| P61981 | YWHAG | 0.013568521 | down-regulated |
| Q12905 | ILF2 | 0.013568521 | down-regulated |
| Q16643 | DBN1 | 0.013568521 | down-regulated |
| P07910 | HNRNPC | 0.013568521 | down-regulated |
| P62913 | RPL11 | 0.013568521 | down-regulated |
| P23396 | RPS3 | 0.013568521 | down-regulated |
| P06576 | ATP5F1B | 0.013568521 | down-regulated |
| P68431 | H3C1 | 0.013568521 | down-regulated |
| Q14103 | HNRNPD | 0.013568521 | down-regulated |
| P55209 | NAP1L1 | 0.013568521 | down-regulated |
| P63010 | AP2B1 | 0.013568521 | down-regulated |
| O14979 | HNRNPDL | 0.013568521 | down-regulated |
| P62241 | RPS8 | 0.013568521 | down-regulated |
| Q9Y3U8 | RPL36 | 0.013568521 | down-regulated |
| P68363 | TUBA1B | 0.013568521 | down-regulated |
| P54577 | YARS | 0.013568521 | down-regulated |
| P35580 | MYH10 | 0.013568521 | down-regulated |
| P38159 | RBMX | 0.013568521 | down-regulated |
| P41219 | PRPH | 0.013568521 | down-regulated |
| Q8TC07 | TBC1D15 | 0.013568521 | down-regulated |
| Q00610 | CLTC | 0.013568521 | down-regulated |
| Q8IZA0 | KIAA0319L | 0.013568521 | down-regulated |
| P04350 | TUBB4A | 0.013568521 | down-regulated |
| Q16629 | SRSF7 | 0.013568521 | down-regulated |
| Q6FHJ7 | SFRP4 | 0.013568521 | down-regulated |
| P84103 | SRSF3 | 0.013568521 | down-regulated |
| P61313 | RPL15 | 0.013568521 | down-regulated |
| P46783 | RPS10 | 0.013568521 | down-regulated |
| P18124 | RPL7 | 0.013568521 | down-regulated |
| P17844 | DDX5 | 0.013568521 | down-regulated |
| Q14194 | CRMP1 | 0.013568521 | down-regulated |
| Q71DI3 | HIST2H3A | 0.013568521 | down-regulated |
| P0DN76 | U2AF1L5 | 0.013568521 | down-regulated |
| P61247 | RPS3A | 0.013568521 | down-regulated |
| Q9UQ80 | PA2G4 | 0.013568521 | down-regulated |
| P16402 | H1-3 | 0.013568521 | down-regulated |
| Q99623 | PHB2 | 0.013568521 | down-regulated |
| P07237 | P4HB | 0.013568521 | down-regulated |
| P14866 | HNRNPL | 0.013568521 | down-regulated |
| O75367 | H2AFY | 0.013568521 | down-regulated |
| P27635 | RPL10 | 0.013568521 | down-regulated |
| O75533 | SF3B1 | 0.013568521 | down-regulated |
| P78371 | CCT2 | 0.013568521 | down-regulated |
| P0DP23 | CALM1 | 0.013568521 | down-regulated |
| P46777 | RPL5 | 0.013568521 | down-regulated |
| P16104 | H2AFX | 0.013568521 | down-regulated |
| P62701 | RPS4X | 0.013568521 | down-regulated |
| P40227 | CCT6A | 0.013568521 | down-regulated |
| Q5EB52 | MEST | 0.013568521 | down-regulated |
| P83731 | RPL24 | 0.013568521 | down-regulated |
| P55884 | EIF3B | 0.013568521 | down-regulated |
| P49588 | AARS | 0.013568521 | down-regulated |
| Q5JXB2 | UBE2NL | 0.013568521 | down-regulated |
| P19338 | NCL | 0.013568521 | down-regulated |
| P62847 | RPS24 | 0.013568521 | down-regulated |
| P60660 | MYL6 | 0.013568521 | down-regulated |
| P11413 | G6PD | 0.013568521 | down-regulated |
| P62081 | RPS7 | 0.013568521 | down-regulated |

### REFERENCES

1. Prof et al. Conformational disease, 1997, The Lancet, vol. 350, pp. 134-138.
2. Westermark et al. Islet amyloid polypeptide, islet amyloid, and diabetes mellitus, 2011, Physiol Rev. vol. 91, pp. 795-826.
3. Hull et al. Islet amyloid: a critical entity in the pathogenesis of type 2 diabetes, 2004, J. Clin. Endocrinol. Metab. vol. 89, pp. 3629-3643.
4. Janson et al. Spontaneous diabetes mellitus in transgenic mice expressing human islet amyloid polypeptide, 1996, Proc Natl Acad Sci USA. vol. 93, pp. 7283-7288.
5. Jurgens et al. β-cell loss and β-cell apoptosis in human type 2 diabetes are related to islet amyloid deposition, 2011, Am J Pathol. vol. 178, pp. 2632-4260.
6. Vogt et al. Anti-IAPP Monoclonal Antibody Improves Clinical Symptoms in a Mouse Model of Type 2 Diabetes, 2021, Vaccines (Basel). vol. 9, pp. 1316.
7. Raimundo et al. Islet Amyloid Polypeptide: A Partner in Crime With AR in the Pathology of Alzheimer's Disease, 2020, Front Mol Neurosci. vol. 13, pp. 35.
8. Soragni et al. A Designed Inhibitor of p53 Aggregation Rescues p53 Tumor Suppression in Ovarian Carcinomas. 2016, Cancer Cell. vol. 9, pp. 90-103.
9. Bykov et al. Restoration of the tumor suppressor function to mutant p53 by a low-molecular-weight compound. 2002, Nat Med. vol. 8, pp. 282-288.
10. Wang et al. The self-interaction of native TDP-43 C terminus inhibits its degradation and contributes to early proteinopathies, 2012, Nature Commun., vol. 3, pp. 766.
11. Kato et al. Cell-free formation of RNA granules: low complexity sequence domains form dynamic fibers within hydrogels, 2012, Cell, vol. 149, pp. 753-767.
12. Han et al. Cell-free formation of RNA granules: bound RNAs identify features and components of cellular assemblies, 2012, Cell, vol. 149, pp. 768-779.
13. Brangwynne et al. Germline P granules are liquid droplets that localize by controlled dissolution/condensation, 2009, Science vol. 324, pp. 1729-1732.

Other embodiments of the invention will be apparent to those skilled in the art from consideration of the specification and practice of the invention disclosed herein. It is intended that the specification and examples be considered as exemplary only.

1. A method for detecting a human disease in a human subject, comprising,
optionally obtaining a biofluid sample from the subject,
adding an isoxazole to the obtained biofluid sample to form a biofluid isoxazole composition in the biofluid sample, and
detecting a presence of the biofluid isoxazole composition.

2. The method of claim 1, wherein the isoxazole is biotin-isoxazole, (6-(5-(Thiophen-2-yl)isoxazole-3-carboxamido)hexyl 5-((3aS,4S,6aR)-2-oxohexahydro-1H-thieno[3,4-d]imidazol-4-yl)pentanoate), or its salt or an analog thereof, especially biotin-isoxazole and very especially biotin-isoxazole, such as OG3 6-(5-(5-chlorothiophen-2-yl)isoxazole-3-carboxamido)hexyl 5-((3aS,4S,6aR)-2-oxohexahydro-1H-thieno[3,4-d]imidazol-4-yl)pentanoate) and OG4 (6-(5-(benzo[b]thiophen-2-yl)isoxazole-3-carboxamido)hexyl5-((3aS,4S,6aR)-2-oxohexahydro-1H-thieno[3,4-d]imidazol-4-yl)pentanoate).

3. The method of claim 1, wherein the biofluid sample is urine, whole blood, plasma, or serum, cerebrospinal fluid (CSF), saliva, or mucosa, such as, urine, saliva, CSF or plasma, and especially CSF or plasma.

4. The method of claim 1, wherein the human disease is prediabetes, diabetes, lung cancer, and pancreatic cancer, Alzheimer's disease (AD), parkinson disease dementia (PD), and dementia with Lewy body (DLB).

5. The method of claim 1, further comprising thereafter treating the human subject for the human disease or thereafter changing an existing treatment of the human subject for the human disease based on the detecting the presents of the biofluid isoxazole composition, such as a treatment including pharmaceutical therapy for the human disease, and optionally further comprising diagnosing the human disease in the human subject.

6. The method of claim 1, wherein the concentration of isoxazole ranges from 0.075 mM to 0.225 mM, preferably from 0.100 mM to 0.200 mM, in the biofluid sample.

7. The method of claim 1, wherein the biofluid isoxazole composition is in a precipitate.

8. The method of claim 7, wherein the human disease is prediabetes, diabetes, lung cancer, and pancreatic cancer, Alzheimer's disease (AD), parkinson disease dementia (PD), and dementia with Lewy body (DLB).

9. The method of claim 1, further comprising adding a polypeptide to biofluid isoxazole composition in the biofluid sample to facilitate detection by an immune assay, such as a blot assay, a chemiluminescence immunoassay, an enzyme-linked immunosorbent assay (ELISA), a light scattering immunoassay, a radiolabeled immunoassay, in particular, ELISA or a Western blot.

10. The method of claim 9, wherein the polypeptide is (a) an antibody, or (b) an immunoglobulin chain, or a binding domain thereof which binds to the biofluid isoxazole composition.

11. The method of claim 9, wherein the human disease is prediabetes, and wherein the polypeptide for detecting prediabetes is an antibody against IAPP, p-TDP-43, amyloid β, α-synuclein, IGKV1-5, SERPINA10, IGHV3-74, APOC3, PARVB, IGHV3-23, CFL1, IGLC2, TTR, SAA4, HBA1, HBB, or CLEC3B.

12. The method of claim 9, wherein the human diseases is diabetes, and wherein the polypeptide for detecting diabetes is an antibody against IAPP, p-TDP-43, amyloid β, α-synuclein, IAPP, phospho-TDP-43, amyloid beta, PLEK, KRT1, IGA2, LBP, IGHV3-30, IGHV4-28, C4B, APOB, AMBP, TUBB, GAPDH, KRT2, KRT10, SERPINA10, IGHV3-74, APOC3, IGHV3-23, IGLC2, CLEC3B, IGKV3-7, IGKV3D-20, IGKV1-6, CD5L, IGHM, IGKV1-17, IGHV5-10-1, or YWHAE.

13. The method of claim 9, wherein the human diseases is lung cancer, and wherein the polypeptide for detecting lung cancer is an antibody against Rb, IGHV5-10-1, ECH1, SRC, GP5, TUBA1A, ZYX, LBP, CTTN, F13A1, HSPB1, GANAB, CD36, HSP90AA1, TGFB1, GDI1, GNAQ, SAA1, CRP, SAA2, TGFB1I1, GPI, ARPC1B, NAP1L1, HPSE, PGAM4, HK1, STXBP2, INF2, CCT6A, BTK, SERPINB1, IGKV6D-21, APOC2, IGFALS, APOC3, APOB, APOA2, ITIH1, ITIH2, TTR, LPA, PON1, HLA-A, APOD, APOA1, SAA4, CLEC3B, AHSG, SERPINA4, AZGP1, SHBG, SERPINA5, ITIH3, TF, HPR, APOE, APOM, IGKV3D-11, GPX3, FCN3, SERPINC1, CPB2, IGKV1-5, RBP4, APOA4, PGLYRP2, IGHV3-30, ALB, C4BPB, AFM, GC, IGHG4, HBA2, SERPINA6, SERPINF2, CLU, LGALS3BP, or KLKB1.

14. The method of claim 9, wherein the human diseases is pancreatic cancer, and wherein the polypeptide for detecting pancreatic cancer is an antibody against p53, C7, C9, F13B, CANX, GANAB, GNAQ, SAA1, CRP, SAA2, TGFB1I1, GPI, ARPC1B, NAP1L1, HPSE, PGAM4, PLTP, IGKV6D-21, APOC2, IGFALS, APOC3, APOB, APOA2, PON1, APOA1, CLEC3B, SHBG, SERPINA5, FCN3, CPB2, IGHV3-30, LGALS3BP, IGG1, IGKV1-39, PF4V1, IGKV1D-33, IGKV2-30, GNB1, YWHAB, RAB1B, SERPINA10, HSPD1, HLA-C, CPN1, IGHV4-4, CAPNS1, HLA-B, TPM1, IGHV1-3, TUBA1A, or TGFB1.

15. The method of claim 9, wherein the human diseases is AD, and wherein polypeptide for detecting AD is an antibody against FN1, LTBP4, PLXNB2, SNED1, AFM, ANK1, SPTB, KLK6, THBS2, ACTBL2, APOL1, SERPINE1, ANXA2, OMG, ACTN1, CD5L, GPLD1, NRCAM, LTBP2, HABP2, BGN, BTD, APOB, CHRD, IGKV3D-15, C4BPB, IGHV5-10-1, HBD, CD109, QSOX2, PLOD1, MEGF10, FGFR1, LMAN1, CA1, CNTNAP4, MAN2A2, PLD3, MRC2, KRT14, GPC1, CD81, STOM, CKB, NCAN, MYOC, SEMA4B, NPTX2, COL3A1, FAM20C, LAMB1, ITIH3, NEGR1, IGKV1-5, LAMC1, FBLN7, COL6A2, SPTA1, B3GNT9, CFHR3, CTSO, LTBP1, LRRC4B, LINGO1, NRXN1, PTPRD, PROC, KRT16, GASKIB, LAMB3, IGHV5-51, FSTL5, SLITRK1, ROBO1, MASP1, TNXB, L1CAM, SEZ6L2, CPXM2, PLAU, TNR, NRXN3, VAT1, MEGF8, BMP6, HRNR, NOTCH3, TMEM132A, H4C1, HNRNPA1, YWHAZ, HNRNPA2B1, HNRNPA3, HNRNPAB, RPSA, MDH1, AP1B1, SNRPD3, RPL12, CAPZA1, ATP6V1B2, NOP58, TARDBP, CCT4, DPYSL3, CAPZB, EEF1G, ARPC3, KPNB1, FAM3C, RACK1, TUBB3, TAGLN2, YWHAQ, HNRNPM, CCT7, SNRNP70, PGAM1, HNRNPUL2, ACTG1, CCT3, GARS1, PABPC1, SND1, DHX9, ANXA5, EFTUD2, ILF3, DDX39B, GDI2, POSTN, RAB10, TUBB4B, SET, RPLP0, HNRNPH2, VSTM2A, FBL, TRA2B, CAND1, H2AC4, TUBB, RAB39B, TPM3, CPB2, RAN, H3-3A, VCP, ELAVL1, TUBA1C, H2BC12, HSPD1, PTGES3, GNB1, EPRS, IGKV1-9, RAP1B, CORO1C, NLGN1, CBX5, VAT1L, RPL7A, PTBP1, ELAVL4, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, HNRNPU, XPO1, CCT8, HNRNPH1, VDAC1, HNRNPF, SSRP1, SLC25A4, PGK1, FUS, YBX1, RPL18A, TARS1, YWHAG, ILF2, DBN1, HNRNPC, RPL11, RPS3, PPIA, ATP5F1B, H3C1, HNRNPD, NAP1L1, AP2B1, RPS9, HNRNPDL, RPS8, RPL36, TUBAIB, YARS, MYH10, RBMX, PRPH, TBC1D15, CLTC, KIAA0319L, CDH2, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, TCP1, RPS24, MYL6, G6PD, or RPS7.

16. The method of claim 9, wherein the human disease is PD, and wherein polypeptide for detecting PD is an antibody against FN1, A2M, CP, SPARCL1, CHL1, RELN, LTBP4, PLXNB2, SNED1, CHGB, AFM, ANK1, KLK6, THBS2, ACTBL2, SEZ6, APOL1, SERPINE1, ANXA2, OMG, APLP2, GPLD1, NRCAM, LTBP2, HABP2, NELL2, BGN, BTD, CHRD, IGKV3D-15, IGHV5-10-1, CD109, QSOX2, PLOD1, MEGF10, FGFR1, LMAN1, CNTNAP4, MAN2A2, PLD3, MRC2, KRT14, GPC1, CKB, NCAN, MYOC, SEMA4B, NPTX2, COL3A1, FAM20C, LAMB1, SEZ6L, NEGR1, LAMC1, FBLN7, COL6A2, B3GNT9, CFHR3, CTSO, LTBP1, LRRC4B, LINGO1, NRXN1, PTPRD, PROC, KRT16, IGHV5-51, SLITRK1, ROBO1, MASP1, L1CAM, SEZ6L2, CPXM2, PLAU, TNR, NRXN3, VAT1, MEGF8, BMP6, HRNR, NOTCH3, TMEM132A, PTPRF, LAMB2, MAN2A1, ACTN1, HPR, IGHV3-9, DHX15, PSMA6, ST13, CFL1, LDHA, H4C1, HSPA8, CSNK2A1, RPL4, TPI1, KRT6B, RPL14, HNRNPA2B1, NOP56, HNRNPA3, EEF2, HNRNPAB, RPSA, MDH1, AP1B1, SNRPD3, RPL12, CAPZA1, ATP6V1B2, NOP58, TARDBP, CCT4, DPYSL3, EEF1G, ARPC3, KPNB1, FAM3C, TUBB3, TAGLN2, YWHAQ, HNRNPM, CCT7, SNRNP70, PGAM1, HNRNPUL2, ACTG1, CCT3, GARS1, SND1, DHX9, ANXA5, EFTUD2, ILF3, DDX39B, GDI2, POSTN, RAB10, TUBB4B, SET, RPLP0, HNRNPH2, FBL, TRA2B, CAND1, H2AC4, TUBB, RAB39B, TPM3, CPB2, RAN, H3-3A, VCP, ELAVL1, TUBA1C, H2BC12, PTGES3, GNB1, EPRS, IGKV1-9, RAP1B, CORO1C, NLGN1, CBX5, VAT1L, RPL7A, PTBP1, TKT, HNRNPK, RARS, SLC25A5, PCBP3, RPL17, MDH2, ACLY, EIF5A, EIF4A1, IGLC7, RUVBL1, MGAT5, EZR, SYNCRIP, GOT1, TUBA1A, EEF1A1, PCBP2, XPO1, HNRNPH1, VDAC1, HNRNPF, SLC25A4, PGK1, FUS, YBX1, RPL18A, TARS1, YWHAG, ILF2, DBN1, HNRNPC, RPL11, RPS3, ATP5F1B, H3C1, HNRNPD, NAP1L1, AP2B1, HNRNPDL, RPS8, RPL36, TUBAIB, YARS, MYH10, RBMX, PRPH, TBC1D15, CLTC, KIAA0319L, TUBB4A, SRSF7, SFRP4, SRSF3, RPL15, RPS10, RPL7, DDX5, CRMP1, HIST2H3A, U2AF1L5, RPS3A, PA2G4, H1-3, PHB2, P4HB, HNRNPL, H2AFY, RPL10, SF3B1, CCT2, CALM1, RPL5, H2AFX, RPS4X, CCT6A, MEST, RPL24, EIF3B, AARS, UBE2NL, NCL, RPS24, MYL6, G6PD, or RPS7.

17. The method of claim 9, wherein the human disease is DLB, and wherein the polypeptide for detecting DLB is an antibody against KRT1, IAPP, IGHG4, IGHV5-10-1, IGHA1, IGA2, FN1, LBP, IGHV4-28, AMBP, IGHV3-48, HLA-A, F10, IGKV1-27, SERPINA10, PARVB, IGLC2, TTR, HBA1, HBB, CLEC3B, CA1, APOA1, STOM, C1QA, CD5L, GP5, IGFALS, CPN1, IGKV1-9, F13A1, IGHM, APOA2, KRT9, SERPINA7, or YWHAE.

18. The method of claim 11, wherein the biofluid sample is urine, whole blood, plasma, or serum, cerebrospinal fluid (CSF), saliva, or mucosa, such as, urine, saliva or plasma, and especially plasma.

19. The method of claim 11, wherein the method is ELISA, such as direct, indirect, sandwich, or competitive ELISA.

20. Use of an isoxazole to detect a human disease in the method of claim 1.

21-86. (canceled)
